SEQUENCE LISTING (61 sequences)

SEQ ID NO: 1 - protein, 448 aa, Artificial
  synthetic peptide
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, Artificial
  synthetic peptide
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, Artificial
  synthetic peptide
QVTLKESGPGILQSSQTLSLTCTFSGFSLSTYGMGVGWIRQPSGKGLEWLTNIWWDDDKYYNSSLKNRLTVSKDTANNQVFLKFTSVDIADTATYYCGRMEWGGSFDYWGQGIMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVAVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 4 - protein, 219 aa, Artificial
  synthetic peptide
DIVMTQTPVSLSVSLGGQVSISCRSSQSLEHRNGITFLSWYLQKPGQSPQLLIYKVSNRFSGISDRFSGRGSGTDFVLQINRVEPDDLGVYYCGQGTHYPLTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - protein, 448 aa, Artificial
  synthetic peptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHYPGGFDYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 6 - protein, 214 aa, Artificial
  synthetic peptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 7 - protein, 447 aa, Artificial
  synthetic peptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 218 aa, Artificial
  synthetic peptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 440 aa, Artificial
  synthetic peptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial
  synthetic peptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 448 aa, Artificial
  synthetic peptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial
  synthetic peptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 450 aa, Artificial
  synthetic peptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 216 aa, Artificial
  synthetic peptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 15 - protein, 451 aa, Artificial
  synthetic peptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 215 aa, Artificial
  synthetic peptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 10 aa, Artificial
  synthetic peptide
GYTFTHYGMN

SEQ ID NO: 18 - protein, 17 aa, Artificial
  synthetic peptide
WVNTYTGEPTYADDFKG

SEQ ID NO: 19 - protein, 9 aa, Artificial
  synthetic peptide
EGEGLGFGD

SEQ ID NO: 20 - protein, 16 aa, Artificial
  synthetic peptide
RSSQSIVHSHGDTYLE

SEQ ID NO: 21 - protein, 7 aa, Artificial
  synthetic peptide
KVSNRFS

SEQ ID NO: 22 - protein, 9 aa, Artificial
  synthetic peptide
FQGSHIPVT

SEQ ID NO: 23 - protein, 5 aa, Artificial
  synthetic peptide
NYYMY

SEQ ID NO: 24 - protein, 17 aa, Artificial
  synthetic peptide
GINPSNGGTNFNEKFKN

SEQ ID NO: 25 - protein, 11 aa, Artificial
  synthetic peptide
RDYRFDMGFDY

SEQ ID NO: 26 - protein, 15 aa, Artificial
  synthetic peptide
RASKGVSTSGYSYLH

SEQ ID NO: 27 - protein, 6 aa, Artificial
  synthetic peptide
LASYLE

SEQ ID NO: 28 - protein, 9 aa, Artificial
  synthetic peptide
QHSRDLPLT

SEQ ID NO: 29 - protein, 5 aa, Artificial
  synthetic peptide
NSGMH

SEQ ID NO: 30 - protein, 17 aa, Artificial
  synthetic peptide
VIWYDGSKRYYADSVKG

SEQ ID NO: 31 - protein, 4 aa, Artificial
  synthetic peptide
NDDY

SEQ ID NO: 32 - protein, 11 aa, Artificial
  synthetic peptide
RASQSVSSYLA

SEQ ID NO: 33 - protein, 7 aa, Artificial
  synthetic peptide
DASNRAT

SEQ ID NO: 34 - protein, 9 aa, Artificial
  synthetic peptide
QQSSNWPRT

SEQ ID NO: 35 - protein, 10 aa, Artificial
  synthetic peptide
GFTFSDSWIH

SEQ ID NO: 36 - protein, 18 aa, Artificial
  synthetic peptide
AWISPYGGSTYYADSVKG

SEQ ID NO: 37 - protein, 9 aa, Artificial
  synthetic peptide
RHWPGGFDY

SEQ ID NO: 38 - protein, 11 aa, Artificial
  synthetic peptide
RASQDVSTAVA

SEQ ID NO: 39 - protein, 7 aa, Artificial
  synthetic peptide
SASFLYS

SEQ ID NO: 40 - protein, 9 aa, Artificial
  synthetic peptide
QQYLYHPAT

SEQ ID NO: 41 - protein, 5 aa, Artificial
  synthetic peptide
SYIMM

SEQ ID NO: 42 - protein, 17 aa, Artificial
  synthetic peptide
SIYPSGGITFYADTVKG

SEQ ID NO: 43 - protein, 11 aa, Artificial
  synthetic peptide
IKLGTVTTVDY

SEQ ID NO: 44 - protein, 14 aa, Artificial
  synthetic peptide
TGTSSDVGGYNYVS

SEQ ID NO: 45 - protein, 7 aa, Artificial
  synthetic peptide
DVSNRPS

SEQ ID NO: 46 - protein, 10 aa, Artificial
  synthetic peptide
SSYTSSSTRV

SEQ ID NO: 47 - protein, 5 aa, Artificial
  synthetic peptide
RYWMS

SEQ ID NO: 48 - protein, 17 aa, Artificial
  synthetic peptide
NIKQDGSEKYYVDSVKG

SEQ ID NO: 49 - protein, 12 aa, Artificial
  synthetic peptide
EGGWFGELAFDY

SEQ ID NO: 50 - protein, 12 aa, Artificial
  synthetic peptide
RASQRVSSSYLA

SEQ ID NO: 51 - protein, 7 aa, Artificial
  synthetic peptide
DASSRAT

SEQ ID NO: 52 - protein, 9 aa, Artificial
  synthetic peptide
QQYGSLPWT

SEQ ID NO: 53 - protein, 9 aa, Artificial
  synthetic peptide
NLVPMVATV

SEQ ID NO: 54 - protein, 444 aa, Artificial
  synthetic peptide
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 214 aa, Artificial
  synthetic peptide
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 8 aa, Artificial
  synthetic peptide
GFTFSNFG

SEQ ID NO: 57 - protein, 8 aa, Artificial
  synthetic peptide
ISGGGRDT

SEQ ID NO: 58 - protein, 10 aa, Artificial
  synthetic peptide
VKWGNIYFDY

SEQ ID NO: 59 - protein, 5 aa, Artificial
  synthetic peptide
LSINT

SEQ ID NO: 60 - protein, 3 aa, Artificial
  synthetic peptide
AAS

SEQ ID NO: 61 - protein, 9 aa, Artificial
  synthetic peptide
QQSSNTPFT

(19) United States
(12) Patent Application Publication
(10) Pub. No.: US 2019/0336496 A1
(43) Pub. Date: Nov. 7, 2019

(54) SELECTIVE BCL-2 INHIBITORS IN COMBINATION WITH AN ANTI-PD-1 OR AN ANTI-PD-L1 ANTIBODY FOR THE TREATMENT OF CANCERS

(71) Applicant: AbbVie Inc., North Chicago, IL (US)

(72) Inventors: Tamar Uziel, Deerfield, IL (US); Joel D. Leverson, Libertyville, IL (US); William N. Pappano, Libertyville, IL (US); Dipica B. Maganbhai Haribhai, North Chicago, IL (US); Rebecca Mathew, North Chicago, IL (US); Fred Kohlhapp, North Chicago, IL (US); Cherie K. Donawho, Burlington, WI (US)

(21) Appl. No.: 16/277,091

(22) Filed: Feb. 15, 2019

Related U.S. Application Data
(60) Provisional application No. 62/763,106, filed on Feb. 16, 2018.
(60) Provisional application No. 62/764,850, filed on Aug. 15, 2018.

(51) Int. Cl. A61K 31/496; A61K 31/5377; C07K 16/28; A61P 35/00

(52) U.S. Cl. CPC A61K 31/496; A61K 31/5377; A61P 35/00; C07K 16/2827; C07K 16/2818

(57) ABSTRACT

This invention pertains to a method for the treatment of cancer in a subject comprising administering to the subject an effective amount of a selective BCL-2 inhibitor or a prodrug or pharmaceutically acceptable salt thereof in combination with an effective amount of an anti-PD-1 antibody or an anti-PD-L1 antibody.

### RELATED APPLICATIONS

This application claims priority from U.S. Provisional Application No. 62/763,106, filed Feb. 16, 2018 and to U.S. Provisional Application No. 62/764,850, filed Aug. 15, 2018. The entire contents of the foregoing applications are expressly incorporated by reference.

### FIELD OF THE INVENTION

This invention pertains to the use of selective BCL-2 inhibitors or prodrugs thereof in combination with either an anti-PD-1 antibody or an anti-PD-L1 antibody in the treatment of hematologic cancers or solid tumor cancers.

### BACKGROUND OF THE INVENTION

The BCL-2 family of proteins are the key regulators of mitochondria-dependent apoptosis in nucleated cells and consists of both anti-apoptotic (BCL-X_{L}, BCL-2, BCL-W, A1, MCL-1) and pro-apoptotic (BAK, BAX, BID, BIM, BAD, BIK, BMF, NOXA, PUMA) members. Cellular expression of anti-apoptotic BCL-2 proteins is associated with inhibition of apoptosis and, in cases of overexpression, can result in aberrant proliferation. Involvement of BCL-2 proteins in a number of cancers is described in PCT Patent Application Publication WO 2005/049593, and PCT Patent Application Publication WO 2005/024636. BCL-2 has long been known as a cancer target and it has been implicated primarily in the survival of hematologic tumors.

Molecules capable of inhibiting anti-apoptotic BCL-2 proteins may increase apoptosis and lead to reduced cell proliferation, thereby leading to improved outcomes related to the treatment and prevention of cancer. Venetoclax (Venclexta™, Venclyxto™, ABT-199) is a selective BCL-2 inhibitor that was recently approved by the FDA for the treatment of patients with chronic lymphocytic leukemia (CLL) or small lymphocytic lymphoma (SLL), with or without 17p deletion, who have received at least one prior therapy. In addition to CLL, early signs of clinical activity have been observed with venetoclax in acute myelogenous leukemia, mantle cell lymphoma, Waldenstrom's macroglobulinemia, follicular lymphoma, diffuse large B-cell lymphomas and multiple myeloma, both as a single agent or in combination with a number of other therapeutic agents (see Ashkenazi, A., Fairbrother, W., Leverson, J. D., and Souers, A. J., From basic apoptosis discoveries to advanced selective BCL-2 family inhibitors. Nature Reviews Drug Discovery 16, 273-284 (2017); and Leverson, J. D., Sampath, D., Souers, A. J., Rosenberg, S. H., Fairbrother, W. J., Amiot, M., Konopleva, M., and Letai, A., Found in translation: how preclinical research is guiding the clinical development of the BCL-2-selective inhibitor venetoclax. Cancer Discovery 7, 1376-1393 (2017)).

Harnessing the body's immune system to detect and destroy tumor cells represents another therapeutic strategy for cancer, and recent years have seen the advance of so-called “checkpoint inhibitors”—molecules targeting proteins on the surface of tumor and/or immune cells that enable tumors to evade detection and elimination by the immune system. By inhibiting these targets, anti-tumor immune responses mediated by cytotoxic T-lymphocytes (CTLs) can be “reinvigorated”, leading to strong efficacy and durable responses. Some checkpoint targets include CTLA4 (cytotoxic T-lymphocyte-associated antigen 4), PD-1 (programmed cell death protein-1) and PD-L1 (programmed death ligand-1), which have been successfully targeted using monoclonal antibodies such as ipilimumab (CTLA4-targeted); nivolumab, cemiplimab, and pembrolizumab (PD-1-targeted); and atezolizumab, durvalumab, and avelumab (PD-L1-targeted) (Topalian, S. L., Drake, C. G., and Pardoll, D. M., Immune Checkpoint Blockade: A Common Denominator Approach to Cancer Therapy. Cancer Cell 27, 450-461 (2015)). These agents are now approved by regulatory agencies for the treatment of various tumor types, including melanoma, Merkel cell carcinoma, lung cancer, renal cell carcinoma, urothelial cancer and unresectable metastatic solid tumors with microsatellite instability or mismatch repair deficiencies.

Clinically and in experimental studies, venetoclax has been shown to induce significant reductions in B-lymphocytes (for example, see Lu, P., Fleischmann, R., Curtis, C., Ignatenko, S., Clarke, S. H., Desai, M., Wong, S. L., Grebe, K. M., Black, K., Zeng, J., Stolzenbach, J., and Medema, J. K., Safety and pharmacodynamics of venetoclax (ABT-199) in a randomized single and multiple ascending dose study in women with systemic lupus erythematosus. Lupus 27, 290-302 (2017). Khaw, S. L., Merino, D., Anderson, M. A., Glaser, S. P., Bouillet, P., Roberts, A. W., and Huang, D. C., Both leukaemic and normal peripheral B lymphoid cells are highly sensitive to the selective pharmacological inhibition of prosurvival Bcl-2 with ABT-199. Leukemia 28, 1207-1215 (2014)) and has also been observed to cause up to 30% reductions in T-cell populations (Lu et al. 2017; Khaw et al. 2014).

In view of the significant reductions in both B-cell and T-cell populations that result from administration of BCL-2 inhibitors, there was a general belief that combination therapy with venetoclax would antagonize the efficacy of checkpoint inhibitors. However, the present inventors have discovered, for the first time, that administration of a therapeutically effective amount of a BCL-2 inhibitor, e.g. venetoclax, with a therapeutically effective amount of either an anti-PD-1 or anti-PD-L1 antibody, results in synergistic regressions of both hematologic cancers as well as solid tumors, thus establishing this combination as a potential treatment option for cancer patients.

### BRIEF SUMMARY OF THE INVENTION

The present invention pertains to methods for the treatment of hematologic cancer in a subject who is in need thereof, comprising administering to the subject an effective amount of either an anti-PD-1 (programmed cell death protein-1) antibody or an anti-PD-L1 (programmed death ligand-1) antibody, in combination with an effective amount of a selective BCL-2 inhibitor or a prodrug or pharmaceutically acceptable salt thereof.

The present invention pertains to methods for the treatment of hematologic cancer in a subject who is in need thereof, comprising administering to the subject an effective amount of either an anti-PD-1 (programmed cell death protein-1) antibody or an anti-PD-L1 (programmed death ligand-1) antibody, in combination with an effective amount of Compound (I), Compound (II), Compound (III) or Compound (IV) or a pharmaceutically acceptable salt thereof.

The present invention pertains to methods for the treatment of a hematologic cancer in a subject who is in need thereof, comprising administering to the subject an effective amount of either an anti-PD-1 (programmed cell death protein-1) antibody or an anti-PD-L1 (programmed death ligand-1) antibody, in combination with an effective amount of Compound (I), wherein Compound (I) is venetoclax or a pharmaceutically acceptable salt thereof.

The present invention pertains to methods for the treatment of hematologic cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof.

The present invention pertains to methods for the treatment of hematologic cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the hematologic cancer is acute lymphoblastic leukemia (ALL), acute myeloid leukemia (AML), chronic myeloid leukemia (CML), chronic lymphocytic leukemia (CLL), small lymphocytic lymphoma (SLL), acute monocytic leukemia (AMoL), Hodgkin's lymphoma, non-Hodgkin lymphomas (NHL), multiple myeloma, or myelodysplastic syndrome.

The present invention pertains to methods for the treatment of hematologic cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the anti-PD-1 antibody is pembrolizumab, nivolumab, cemiplimab, or ABBV-181.

The present invention pertains to methods for the treatment of hematologic cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the anti-PD-1 antibody has a heavy chain sequence comprising SEQ ID NO: 7, and a light chain sequence comprising SEQ ID NO: 8.

The present invention pertains to methods for the treatment of hematologic cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the anti-PD-1 antibody has a heavy chain sequence comprising SEQ ID NO: 9, and a light chain sequence comprising SEQ ID NO: 10.

The present invention pertains to methods for the treatment of hematologic cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the anti-PD-1 antibody has a heavy chain sequence comprising SEQ ID NO: 1, and a light chain sequence comprising SEQ ID NO: 2.

The present invention pertains to methods for the treatment of hematologic cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the anti-PD-1 antibody has a heavy chain sequence comprising SEQ ID NO: 54, and a light chain sequence comprising SEQ ID NO: 55.

The present invention pertains to methods for the treatment of hematologic cancer in a subject who is in need thereof, comprising administering to the subject an effective amount of an anti-PD-L1 (programmed death ligand-1) antibody, in combination with an effective amount of Compound (I), wherein Compound (I) is venetoclax or a pharmaceutically acceptable salt thereof.

The present invention pertains to methods for the treatment of hematologic cancer in a subject who is in need thereof, comprising administering to the subject an effective amount of an anti-PD-L1 (programmed death ligand-1) antibody, in combination with an effective amount of Compound (I), wherein Compound (I) is venetoclax or a pharmaceutically acceptable salt thereof, wherein the hematologic cancer is acute lymphoblastic leukemia (ALL), acute myeloid leukemia (AML), chronic myeloid leukemia (CML), chronic lymphocytic leukemia (CLL), small lymphocytic lymphoma (SLL), acute monocytic leukemia (AMoL), Hodgkin's lymphoma, non-Hodgkin lymphomas (NHL), multiple myeloma, or myelodysplastic syndrome.

The present invention pertains to methods for the treatment of hematologic cancer in a subject who is in need thereof, comprising administering to the subject an effective amount of an anti-PD-L1 (programmed death ligand-1) antibody, in combination with an effective amount of Compound (I), wherein Compound (I) is venetoclax or a pharmaceutically acceptable salt thereof, wherein the anti-PD-L1 antibody is atezolizumab, avelumab, or durvalumab.

The present invention pertains to methods for the treatment of hematologic cancer in a subject who is in need thereof, comprising administering to the subject an effective amount of an anti-PD-L1 antibody, in combination with an effective amount of Compound (I), wherein Compound (I) is venetoclax or a pharmaceutically acceptable salt thereof, wherein the anti-PD-L1 antibody has a heavy chain sequence comprising SEQ ID NO: 11, and a light chain sequence comprising SEQ ID NO: 12.

The present invention pertains to methods for the treatment of hematologic cancer in a subject who is in need thereof, comprising administering to the subject an effective amount of an anti-PD-L1 antibody, in combination with an effective amount of Compound (I), wherein Compound (I) is venetoclax or a pharmaceutically acceptable salt thereof, wherein the anti-PD-L1 antibody has a heavy chain sequence comprising SEQ ID NO: 13, and a light chain sequence comprising SEQ ID NO: 14.

The present invention pertains to methods for the treatment of hematologic cancer in a subject who is in need thereof, comprising administering to the subject an effective amount of an anti-PD-L1 antibody, in combination with an effective amount of Compound (I), wherein Compound (I) is venetoclax or a pharmaceutically acceptable salt thereof, wherein the anti-PD-L1 antibody has a heavy chain sequence comprising SEQ ID NO: 15, and a light chain sequence comprising SEQ ID NO: 16.

The present invention pertains to methods for the treatment of solid tumor cancer in a subject who is in need thereof, comprising administering to the subject an effective amount of an anti-PD-1 (programmed cell death protein-1) antibody, or an anti-PD-L1 (programmed death ligand-1) antibody, in combination with an effective amount of a selective BCL-2 inhibitor or pharmaceutically acceptable salt thereof.

The present invention pertains to methods for the treatment of solid tumor cancer in a subject who is in need thereof, comprising administering to the subject an effective amount of an anti-PD-1 (programmed cell death protein-1) antibody, or an anti-PD-L1 (programmed death ligand-1) antibody in combination with an effective amount of an effective amount of Compound (I), Compound (II), Compound (III), or Compound (IV) or a pharmaceutically acceptable salt thereof.

The present invention pertains to methods for the treatment of a solid tumor cancer in a subject who is in need thereof, comprising administering to the subject an effective amount of an anti-PD-1 (programmed cell death protein-1) antibody, or an anti-PD-L1 (programmed death ligand-1) antibody, in combination with an effective amount of Compound (I), wherein Compound (I) is venetoclax or a pharmaceutically acceptable salt thereof.

The present invention pertains to methods for the treatment of solid tumor cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof.

The present invention pertains to methods for the treatment of solid tumor cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the solid tumor cancer is non-small cell lung cancer, gastric cancer, melanoma, microsatellite instability-high cancer, head and neck squamous cell cancer, metastatic cutaneous squamous cell carcinoma, locally advanced cutaneous squamous-cell carcinoma, urothelial bladder cancer, colorectal cancer, liver cancer, renal cell carcinoma, breast cancer, cervical cancer or Merkel cell carcinoma.

The present invention pertains to methods for the treatment of solid tumor cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the anti-PD-1 antibody is pembrolizumab, nivolumab, cemiplimab, or ABBV-181.

The present invention pertains to methods for the treatment of solid tumor cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the anti-PD-1 antibody has a heavy chain sequence comprising SEQ ID NO: 7, and a light chain sequence comprising SEQ ID NO: 8.

The present invention pertains to methods for the treatment of solid tumor cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the anti-PD-1 antibody has a heavy chain sequence comprising SEQ ID NO: 9, and a light chain sequence comprising SEQ ID NO: 10.

The present invention pertains to methods for the treatment of solid tumor cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the anti-PD-1 antibody has a heavy chain sequence comprising SEQ ID NO: 1, and a light chain sequence comprising SEQ ID NO: 2.

The present invention pertains to methods for the treatment of solid tumor cancer in a subject who is in need thereof, comprising administering to the subject an effective amount of an anti-PD-1 (programmed cell death protein-1) antibody, in combination with an effective amount of Compound (I), wherein Compound (I) is venetoclax or a pharmaceutically acceptable salt thereof, wherein the anti-PD-1 antibody has a heavy chain sequence comprising SEQ ID NO: 54, and a light chain sequence comprising SEQ ID NO: 55.

The present invention pertains to methods for the treatment of solid tumor cancer in a subject who is in need thereof, comprising administering to the subject an effective amount of an anti-PD-L1 (programmed death ligand-1) antibody, in combination with an effective amount of Compound (I), wherein Compound (I) is venetoclax or a pharmaceutically acceptable salt thereof.

The present invention pertains to methods for the treatment of solid tumor cancer in a subject who is in need thereof, comprising administering to the subject an effective amount of an anti-PD-L1 (programmed death ligand-1) antibody, in combination with an effective amount of Compound (I), wherein Compound (I) is venetoclax or a pharmaceutically acceptable salt thereof, wherein the solid tumor cancer is non-small cell lung cancer, gastric cancer, melanoma, microsatellite instability-high cancer, head and neck squamous cell cancer, metastatic cutaneous squamous cell carcinoma, locally advanced cutaneous squamous-cell carcinoma, urothelial bladder cancer, colorectal cancer, liver cancer, renal cell carcinoma, breast cancer, cervical cancer or Merkel cell carcinoma.

The present invention pertains to methods for the treatment of solid tumor cancer in a subject who is in need thereof, comprising administering to the subject an effective amount of an anti-PD-L1 (programmed death ligand-1) antibody, in combination with an effective amount of Compound (I), wherein Compound (I) is venetoclax or a pharmaceutically acceptable salt thereof, wherein the anti-PD-L1 antibody is atezolizumab, avelumab, or durvalumab.

The present invention pertains to methods for the treatment of solid tumor cancer in a subject who is in need thereof, comprising administering to the subject an effective amount of an anti-PD-L1 antibody, in combination with an effective amount of Compound (I), wherein Compound (I) is venetoclax or a pharmaceutically acceptable salt thereof, wherein the anti-PD-L1 antibody has a heavy chain sequence comprising SEQ ID NO: 11, and a light chain sequence comprising SEQ ID NO: 12.

The present invention pertains to methods for the treatment of solid tumor cancer in a subject who is in need thereof, comprising administering to the subject an effective amount of an anti-PD-L1 antibody, in combination with an effective amount of Compound (I), wherein Compound (I) is venetoclax or a pharmaceutically acceptable salt thereof, wherein the anti-PD-L1 antibody has a heavy chain sequence comprising SEQ ID NO: 13, and a light chain sequence comprising SEQ ID NO: 14.

The present invention pertains to methods for the treatment of solid tumor cancer in a subject who is in need thereof, comprising administering to the subject an effective amount of an anti-PD-L1 antibody, in combination with an effective amount of Compound (I), wherein Compound (I) is venetoclax or a pharmaceutically acceptable salt thereof, wherein the anti-PD-L1 antibody has a heavy chain sequence comprising SEQ ID NO: 15, and a light chain sequence comprising SEQ ID NO: 16.

The present invention pertains to methods for the treatment of hematologic cancer in a subject who is in need thereof, consisting of administering to the subject an effective amount of either an anti-PD-1 (programmed cell death protein-1) antibody or an anti-PD-L1 (programmed death ligand-1) antibody, in combination with Compound (I), wherein Compound (I) is venetoclax or a pharmaceutically acceptable salt thereof. The present invention also pertains to methods for the treatment of solid tumor cancer in a subject who is in need thereof, consisting of administering to the subject an effective amount of either an anti-PD-1 (programmed cell death protein-1) antibody or an anti-PD-L1 (programmed death ligand-1) antibody in combination with Compound (I), wherein Compound (I) is venetoclax or a pharmaceutically acceptable salt thereof.

### BRIEF DESCRIPTION OF THE DRAWINGS

FIG. 1: In vitro sensitivity of CT26, EMT6 and MC38 cells to Compound (I). FIG. 1 shows that the cell lines from mouse syngeneic models of solid tumors, CT26, MC38 (both colorectal carcinoma) and EMT6 (breast cancer), are resistant to Compound (I) in vitro. CT26, MC38 and EMT6 cells were incubated with increasing doses of Compound (I) and viability was measured using CellTiter®-Glo (Promega) after 72 hours as described in Example 1.

FIG. 2: In vivo sensitivity of lymphocytes to Compound (I). FIG. 2 shows that mouse lymphoctyes in the lymph node, spleen, and blood, including B-cells, CD8⁺ T-cells, and CD4⁺ T-cells, are sensitive to in vivo treatment with Compound (I), as described in Example 1. Characterization of T-cell subsets by flow cytometry indicates that the fraction of CD4+ and CD8+ effector memory T-cells (TEMs) increases following in vivo treatment with Compound (I). Descriptions of each sub-section are as follows: (A) In vivo sensitivity of lymphocytes following one week of treatment with Compound (I). Naïve CB6F1 mice were treated daily with Compound (I) (50/mg/kg) for one week. Lymph nodes were collected, and immune cells were quantified by flow cytometry; (B) In vivo sensitivity of splenocytes following one week of treatment with Compound (I). Naïve C57BL/6 mice were treated daily with Compound (I) (50/mg/kg) for one week. Spleens were collected and immune cells were quantified by flow cytometry. Mouse splenocytes, including B-cells, CD8+ T-cells, and CD4+ T-cells, were sensitive to in vivo treatment with Compound (I), as described in Example 1; (C) In vivo sensitivity of T-cell subsets in splenocytes following one week of treatment with Compound (I). CD4+ naïve T-cells (TN) and CD8+ central memory T-cells in the spleen from C57BL/6 mice were slightly sensitive to in vivo Compound (I) treatment while the proportion of CD4+ and CD8+ effector memory T-cells (TEM) was increasing; and (D) In vivo sensitivity of T-cell subsets in the blood following one week of treatment with Compound (I). The proportion of CD4+ and CD8+ effector memory T cells (TEM) increased in the blood from C57BL/6 mice following in vivo Compound (I) treatment.

FIG. 3: Anti-tumor effect of Compound (I), anti-PD-1, and Compound (I)-anti-PD-1 combination in CT26 mouse colon carcinoma syngeneic model. FIG. 3 shows tumor growth rate in the CT26 mouse colon carcinoma syngeneic model treated with Compound (I), anti-PD-1 antibody, and the combination of Compound (I), venetoclax, and anti-PD-1 antibody as described in Example 1 and Example 1A. The survival curves of mice inoculated with CT26 tumors and treated with the previously indicated agents are shown. Descriptions of each sub-section are as follows: (A) Tumor growth rate following treatment with venetoclax, anti-PD-1 and venetoclax-anti-PD-1 combination. Each point of the curve represents the mean of 10 tumors. Error bars depict the standard error of the mean; and (B) Survival curves of mice inoculated with CT26 tumors and treated with the indicated agents.

FIG. 4: Characterization of T-cells from spleens of CT26 mice that had been treated +/−Compound (I)-anti-PD-1 combination and re-challenged with tumor cells. FIG. 4 shows an increase in the number of CD8⁺ T-cells with an effector memory phenotype (CD8⁻CD62L⁺CD44⁺) in the spleen of complete responder mice that had been re-inoculated with CT26 cells (group 3) as described in Example 1A. Mice re-challenged with CT26 tumor cells (group 3) mounted an effector memory response compared to non-tumor bearing mice (group 1) and primary CT26-challenged mice (group 2). Group 1: naïve mice, non-tumor-bearing. Group 2: CT26 tumor-bearing mice. Group 3: mice that had CT26 tumors and had responded to anti-PD-1 + venetoclax, and rejected CT26 tumors. Descriptions of each sub-section are as follows: (A) Total CD8⁺ T-cells; (B) Activated CD8⁺ T-cells (CD8⁻CD69⁺); (C) Naïve CD8⁺ T-cells (CD8⁺ CD62L⁺ CD44⁻); and (D) Effector memory CD8⁻ T-cells (CD8⁺ CD62L⁻CD44⁺).

FIG. 5: In vitro T-cell activation following tumor re-challenge using splenocytes from CT26 mice treated +/−Compound (I)-anti-PD-1 combination. FIG. 5 shows that splenocytes from complete responder mice previously treated with a combination of Compound (I) and an anti-PD-1 antibody retain the ability for T-cell activation following tumor re-challenge as measured by IFNγ secretion (FIG. 5A) as described in Example 1A. Maintenance of a specific anti-CT26 memory is demonstrated by a recall response to irradiated CT26 cells (the original tumor) but not with irradiated EMT6 cells (FIG. 5B). Group 1: naïve mice, non-tumor-bearing. Group 2: CT26 tumor-bearing mice. Group 3: mice that had CT26 tumors and had responded to anti-PD-1 + venetoclax, and rejected CT26 tumors.

FIG. 6: Anti-tumor effect of Compound (I), anti-PD-L1, and Compound (I)-anti-PD-L1 combination in EMT6, mouse mammary carcinoma, syngeneic model. FIG. 6 shows tumor growth rate in the EMT6 mouse mammary carcinoma syngeneic model with Compound (I), anti-PD-L1 antibody, and the combination of Compound (I) and anti-PD-L1 antibody as described in Example 1B. The survival curves of mice inoculated with EMT6 tumors and treated with the previously indicated agents are shown. Descriptions of each sub-section are as follows: (A) Tumor growth rate following treatment with venetoclax, anti-PD-L1 and venetoclax-anti-PD-L1 combination. Each point of the curve represents the mean of 10 tumors. Error bars depict the standard error of the mean; and (B) Survival curves of mice inoculated with EMT6 tumors and treated with the indicated agents.

FIG. 7: In vitro sensitivity of human PBMCs to Compound (I) (venetoclax). FIG. 7 shows a dose dependent decrease in the number of B-cells and T-cells in unstimulated human PBMCs when treated with Compound (I) in vitro as described in Example 2. However, T-cells in stimulated PBMCs were not sensitive to treatment with Compound (I). Unstimulated (A) and CD3/CD28 stimulated (B) PBMCs were cultured for 72 hours followed by 24 hours treatment with Compound (I) and immune cells were quantified by flow cytometry. In a separate experiment (C) PBMCs were cultured for 24 hours in the absence/presence of Compound (I). Descriptions of each sub-section are as follows: (A) The number of B and T cells decreased following Compound (I) treatment in a dose-dependent manner in unstimulated conditions in 3 donors; (B) Venetoclax did not affect the number of T cells in stimulated conditions; and (C) The number of B and T cells decreased following Compound (I) treatment in a dose-dependent manner in unstimulated conditions in 9 donors.

FIG. 8: Human naïve T-cells are susceptible to Compound (I) while memory T-cells are not. FIG. 8 shows the in vitro sensitivity of unstimulated (naïve) CD4⁺ T-cells and CD8⁺ T-cells to Compound (I) while CD4⁻ memory T cells are less sensitive as described in Example 2. Under CD3/CD28 stimulated conditions, Compound (I) did not affect the proportion of T-cell subsets. CD8⁺ and CD4⁺ T-cells from FIG. 7 were further analyzed for naïve (TN), central (TCM), effector (TEM) and terminally differentiated (TEMRA) memory T-cell subsets. Descriptions of each sub-section are as follows: (A) Unstimulated conditions—Venetoclax induced a dose-dependent decrease in the proportion of naïve CD4⁺ T-cells and an increase in the proportion of CD4⁺ TCM cells relative to the total number of surviving T-cells (3 donors from FIGS. 7A and 7B); (C) Unstimulated conditions—Venetoclax induced a dose-dependent decrease in the proportion of naïve CD8⁺ T-cells and an increase in the proportion of CD8⁺ TEM and TEMRA cells relative to the total number of surviving T-cells (3 donors from FIGS. 7A and 7B); (B and D) Stimulated conditions—Venetoclax did not affect the proportion of CD4⁻ or CD8⁺ T-cell subpopulations relative to the total number of surviving T-cells after T-cells have been activated in vitro (3 donors from FIGS. 7A and 7B); (E) Unstimulated conditions—of Compound (I) induces a decrease in naïve CD8+ and CD4+ T-cells (TN) and an increase in effector memory T cells (TEM) and terminally differentiated effector memory cells (TEMRA) (average of 9 donors from FIG. 7C); (F and G) Unstimulated conditions—Dose response of Compound (I) of CD4+ and CD8+ TN and TEM T-cells for all 9 donors.

FIG. 9: In vitro sensitivity of T-cell subsets from mouse splenocytes to Compound (I). Naïve T-cells are susceptible to Compound (I) while memory T-cells are not. FIG. 9 shows that in mouse splenocytes both CD4⁺ and CD8⁺ naïve T-cells are sensitive to in vitro Compound (I) treatment while memory T-cells are less sensitive as described in Example 2. BALB/c and C57BL/6 splenocytes were treated with venetoclax for 24 hours, in vitro. Descriptions of each sub-section are as follows: (A) Venetoclax induced a dose-dependent decrease in the proportion of naïve CD4⁺ T-cells and an increase in the proportion of CD4⁺ TCM cells relative to the total number of surviving T-cells; and (B) Venetoclax induced a dose-dependent decrease in the proportion of naïve CD8⁺ T-cells and an increase in the proportion of CD8⁺ effector memory (TEM) cells relative to the total number of surviving T-cells.

FIG. 10: Compound (I) (venetoclax) limits the number of live lymphocytes in human cytomegalovirus-positive (CMV⁺) donors in a CMV recall assay, but spares IFNγ and IL2 producing cells in CD8 T-cells. FIG. 10 shows a decrease in percentage of live cells in a cytomegalovirus (CMV) recall assay in the presence of Compound (I) (venetoclax) in PBMCs from CMV⁺ donors as described in Example 3. Also shown is IFNγ and IL2 production by CD8⁺ T-cells in the presence of Compound (I) or in the presence of Compound (I) and an anti-PD-1 antibody (nivolumab). Furthermore, venetoclax did not impair the activity of antigen specific CMV+ CD8 T cells. Descriptions of each sub-section are as follows: (A) A decrease in percentage of live cells in a CMV recall assay in the presence of Compound (I) was observed; (B) IFNγ production from surviving CD8+ T-cells stimulated with CMV antigen and rechallenged with PMA/ionomycin produced higher levels of IFNγ following venetoclax treatment; (C) IL2 production from surviving CD8+ T-cells stimulated with CMV antigen and rechallenged with PMA/ionomycin produced higher levels of IL-2 following venetoclax treatment. (MFI, mean fluorescence intensity; US, unstimulated; DMSO, drug-free control); (D) A decrease in percentage of live cells in a CMV recall assay in the presence of Compound (I) or Compound (I) and an anti-PD-1 antibody was observed; (E) Surviving CD8+ T-cells stimulated with CMV antigen generate equivalent IFNγ after venetoclax alone or venetoclax and anti-PD-1 treatment. US, unstimulated; DMSO, drug-free control; (F and G) Percent of live CD8+ T-cells measured following their overnight incubation with T2 cells alone or T2 cells loaded with MART control peptide or CMV peptide with or without venetoclax. CMV+CD8+ T-cells were gated and IFNγ secretion was measured by flow cytometry to evaluate function of antigen specific T cell (live CD3+CD8+IFNγ+T cells). The results indicated that the response of CD8+T2 cells was CMV specific and was not impaired by venetoclax. CD8+T2 denotes CD8+ T-cells incubated with T2 cells alone; CD8+T2^{MART} or CD8+T2^{CMV} denote CD8+ T-cells incubated with control peptide MART, or CMV peptide, respectively; (H) Venetoclax did not impair IFNγ secretion in surviving CD8+T2^{CMV} cells. T2 denotes T2 cells alone; CD8+ denotes CMV specific CD8+ T-cells alone.

FIG. 11: BCL-2 inhibitors limit the number of T-cells in a MLR, but do not antagonize IFNγ production and anti-PD-1 response. FIG. 11 shows a mixed lymphocyte reaction (MLR) wherein the anti-PD-1 antibody increases both the cell number and the proportion of CD4⁺ T-cells that produce IFNγ as described in Example 4. Compound (I), venetoclax, reduced the number of CD4⁺ T-cells. However, there was a significant increase in the proportion of CD4⁺ T-cells that produce IFNγ when Compound (I) was added with either the isotype control or an anti-PD-1 antibody. Representative flow cytometry data are also shown. Each mixed lymphocyte reaction shows a combination treatment of venetoclax with either anti-PD-1 or isotype control antibody. Descriptions of each sub-section are as follows: (A and B) T-cell numbers; (C and D) Proportion of CD3⁺ T-cells producing IFNγ (related to A and B); (E and F) Representative flow cytometry plots showing IFNγ⁺ CD3⁺ T-cells; (G) T Cell numbers were reduced upon treatment with various BCL-2 inhibitors alone or in combination with anti-PD-1 antibodies; (H) Proportion of CD3+ T-cells producing IFNγ (related to G); and (I) Quantity of secreted IFNγ (related to G).

FIG. 12. Anti-tumor effect of Compound (I)-anti-PD-1 or -anti-PD-L1 combination in MC38, colon carcinoma, syngeneic model in immune competent and deficient mice. FIG. 12 shows tumor growth rate in the MC38 mouse colon carcinoma syngeneic model with Compound (I), anti-PD-1 or anti-PD-L1 antibodies, and the combination of Compound (I) with anti-PD-1 or anti-PD-L1 antibodies in immune competent (C57BL/6) and immune deficient (SCID) mice as described in Example 1C. The survival curves of mice inoculated with MC38 tumors and treated with the previously indicated agents are shown. Descriptions of each sub-section are as follows: (A) Tumor growth rate following treatment with venetoclax, anti-PD-1 and venetoclax-anti-PD-1 combination in immune competent, C57BL/6, mice. Each point of the curve represents the mean of 10 tumors. Error bars depict the standard error of the mean; (B) Survival curves of mice inoculated with MC38 tumors and treated with the indicated agents (related to Figure A); (C) Tumor growth rate following treatment with venetoclax, anti-PD-L1 and venetoclax-anti-PD-L1 combination in immune competent, C57BL/6, mice. Each point of the curve represents the mean of 10 tumors. Error bars depict the standard error of the mean; (D) Survival curves of mice inoculated with MC38 tumors and treated with the indicated agents (related to Figure C); (E) Tumor growth rate following treatment with venetoclax, anti-PD-1 and venetoclax-anti-PD-1 combination in immune deficient, SCID, mice. Each point of the curve represents the mean of 10 tumors. Error bars depict the standard error of the mean; (F) Tumor growth rate following treatment with venetoclax, anti-PD-L1 and venetoclax-anti-PD-L1 combination in immune deficient, SCID, mice. Each point of the curve represents the mean of 10 tumors. Error bars depict the standard error of the mean.

FIG. 13. Effect of Compound (I) (venetoclax) and Compound (IV) on T-cell subsets in human healthy subjects. FIG. 13 shows immune-phenotyping of T-cell subsets in healthy human subjects following one dose of Compound (I) or one dose of Compound (IV). Subjects were administered with one dose of 100 mg of Compound (I) or Compound (IV) and T-cell subsets in the blood were characterized by flow cytometry one day pre- and seven days post-dosing, respectively. Compounds (I) or (IV) induced a decrease in naïve CD8+ T-cells (TN) and CD4+ and CD8+ central memory T cells (TCM) and an increase in CD4+ and CD8+ effector memory T-cells (TEM) and terminally differentiated effector memory cells (TEMRA) in most of the subjects.

### DETAILED DESCRIPTION OF THE INVENTION

Despite expectations that combining venetoclax with a checkpoint inhibitor would result in reduced efficacy, it has surprisingly been found that venetoclax does not antagonize the immune-mediated anti-tumor activity of anti-PD-1 or anti-PD-L1 antibodies and can instead enhance efficacy in combination therapy. In a CT26 model, the combination therapy of venetoclax with an anti-PD-1 antibody resulted in an increase in tumor growth inhibition (TGI) over treatment with the anti-PD-1 antibody alone, from 32% to 49%. The combination therapy of venetoclax with an anti-PD-1 antibody also resulted in an increase in the number of complete responders over subjects treated with the anti-PD-1 antibody alone, from 0/10 to 3/10. Similar effects were observed for the combination therapy of venetoclax with an anti-PD-L1 antibody in an EMT6 model (TGI increase from 89% to 94%, increase in complete responders from 3/10 to 9/10). Additional similar effects were observed for the combination of venetoclax with the anti-PD-1 or anti-PD-L1 antibodies in an MC38 model (TGI increases from 32% to 73% and 45% to 80%, respectively; increase in complete responders from 1/10 to 2/10 and 0 to 6/10, respectively).

Furthermore, tumor re-challenge experiments showed that venetoclax treatment surprisingly did not interfere with the establishment of anti-tumor memory, because mice responsive to the combination treatment of venetoclax with anti-PD-1 or anti-PD-L1 antibodies actually had developed anti-tumor memory. All of the complete responders to the combination therapy of venetoclax with an anti-PD-1 antibody, when re-inoculated with CT26 tumor cells, mounted an immune-mediated anti-tumor response and tumor engraftment did not occur. Likewise, no tumor engraftment was observed in complete responders for mice treated with a combination of venetoclax with either an anti-PD-1 or anti-PD-L1 antibody when re-challenged with MC38 tumor cells. The treatment of MC38 with venetoclax in combination with an anti-PD-1 or anti-PD-L1 antibody demonstrated that the response was immune-mediated because no TGI was observed in SCID mice.

Moreover, the combination therapy of venetoclax with an anti-PD-1 or anti-PD-L1 antibody unexpectedly demonstrated efficacy in solid, non-hematological tumors. While BCL-2 has primarily been implicated in the survival of hematologic tumors, experiments described herein showed that the combination of venetoclax with an anti-PD-1 or anti-PD-L1 antibody demonstrated efficacy in colon carcinoma and mammary carcinoma models.

In one embodiment, the present invention relates to Compound (I) an inhibitor of BCL-2 protein. It is disclosed in PCT Patent Application Publication WO 2010/138588, incorporated herein by reference in its entirety and for all purposes.

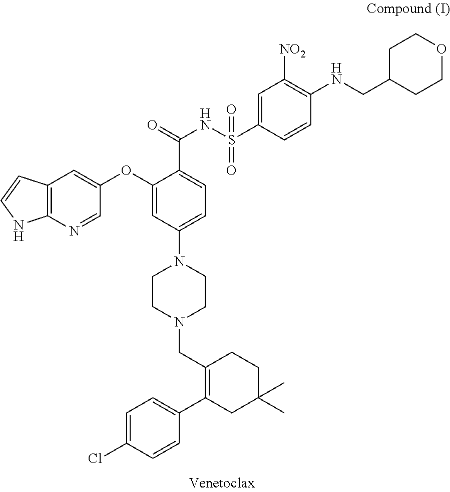 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

In one embodiment, the present invention relates to Compound (II), an inhibitor of BCL-2 protein. It is disclosed in PCT Patent Application Publication WO 2013/110890, incorporated herein by reference in its entirety and for all purposes.

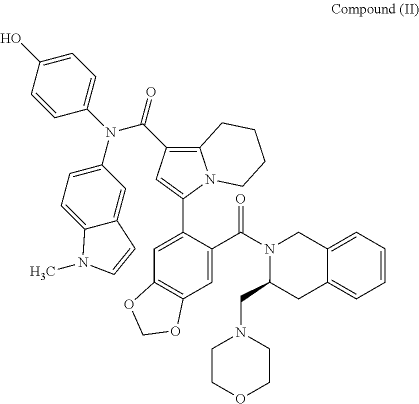 [SMILES: Cn1ccc2cc(N(C(=O)c3cc(-c4cc5c(cc4C(=O)N4Cc6ccccc6C[C@H]4CN4CCOCC4)OCO5)n4c3CCCC4)c3ccc(O)cc3)ccc21]

In one embodiment, the present invention relates to Compound (III), an inhibitor of BCL-2 protein. It is disclosed in PCT Patent Application Publication WO 2013/110890 and Casara, P., Davidson, J., et al. S55746 is a novel orally active BCL-2 selective and potent inhibitor that impairs haematological tumor growth. Oncotarget 9 (28), 20075-20088 (2018), incorporated herein by reference in its entirety and for all purposes.

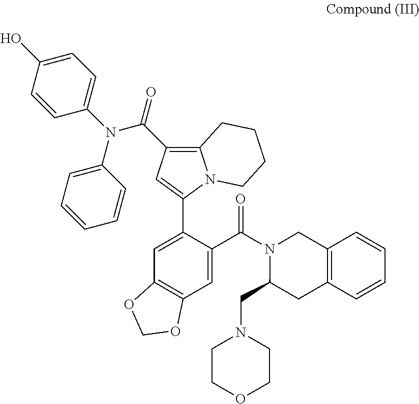 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1]

In one embodiment, the present invention relates to Compound (IV), a water-soluble methylene phosphate prodrug of Compound (I). It has shown pre-clinically rapid conversion to parent active pharmaceutical ingredient (API) by alkaline phosphatases in the gut after oral dosing to provide venetoclax exposures that will be effective in treatment of the full range of current venetoclax indications. It is disclosed in PCT Patent Application Publication WO 2011/150016, incorporated herein by reference in its entirety and for all purposes.

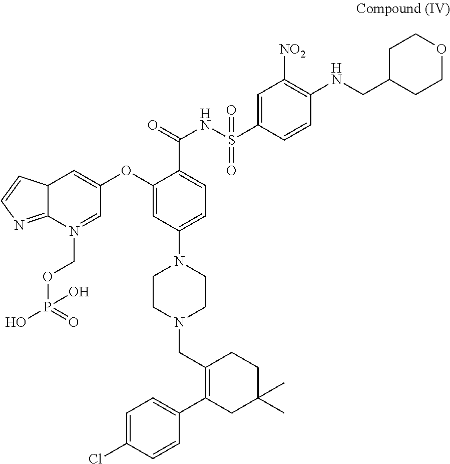

In one embodiment, the present invention relates to pembrolizumab, nivolumab, cemiplimab, or ABBV-181, antibodies with specificity for PD-1 (programmed cell death protein-1). Pembrolizumab is comprised of the heavy chain sequence of SEQ ID NO: 7 and the light chain sequence of SEQ ID NO: 8. Nivolumab is comprised of the heavy chain sequence of SEQ ID NO: 9 and the light chain sequence of SEQ ID NO: 10. Cemiplimab is comprised of the heavy chain sequence of SEQ ID NO: 54 and the light chain sequence of SEQ ID NO: 55. ABBV-181 is comprised of the heavy chain sequence of SEQ ID NO: 1 and the light chain sequence of SEQ ID NO: 2.

In one embodiment, the present invention relates to atezolizumab, avelumab, or durvalumab, antibodies with specificity for PD-L1 (programmed death ligand-1). Atezolizumab is comprised of the heavy chain sequence of SEQ ID NO: 11 and the light chain sequence of SEQ ID NO: 12. Avelumab is comprised of the heavy chain sequence of SEQ ID NO: 13 and the light chain sequence of SEQ ID NO: 14. Durvalumab is comprised of the heavy chain sequence of SEQ ID NO: 15 and the light chain sequence of SEQ ID NO: 16.

In one embodiment, the present invention relates to methods for the treatment of hematologic cancer in a subject who is in need thereof, comprising administering to the subject an effective amount of either an anti-PD-1 (programmed cell death protein-1) antibody or an anti-PD-L1 (programmed death ligand-1) antibody in combination with a selective BCL-2 inhibitor or pharmaceutically acceptable salt thereof.

In one embodiment, the present invention relates to methods for the treatment of hematologic cancer in a subject who is in need thereof, comprising administering to the subject an effective amount of either an anti-PD-1 (programmed cell death protein-1) antibody or an anti-PD-L1 (programmed death ligand-1) antibody in combination with Compound (I), Compound (II), Compound (III), or Compound (IV) or a pharmaceutically acceptable salt thereof.

In one embodiment, the present invention relates to methods for the treatment of hematologic cancer in a subject who is in need thereof, comprising administering to the subject an effective amount of either an anti-PD-1 (programmed cell death protein-1) antibody or an anti-PD-L1 (programmed death ligand-1) antibody in combination with Compound (I), wherein Compound (I) is venetoclax or a pharmaceutically acceptable salt thereof.

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof.

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, and the hematologic cancer is acute lymphoblastic leukemia (ALL), acute myeloid leukemia (AML), chronic myeloid leukemia (CML), chronic lymphocytic leukemia (CLL), small lymphocytic lymphoma (SLL), acute monocytic leukemia (AMoL), Hodgkin's lymphoma, non-Hodgkin lymphomas, multiple myeloma, or myelodysplastic syndrome (MDS).

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is pembrolizumab, nivolumab, cemiplimab, or ABBV-181.

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the heavy chain sequence of SEQ ID NO: 7 and the light chain sequence of SEQ ID NO: 8.

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the heavy chain sequence of SEQ ID NO: 9 and the light chain sequence of SEQ ID NO: 10.

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the heavy chain sequence of SEQ ID NO: 54 and the light chain sequence of SEQ ID NO: 55.

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the heavy chain sequence of SEQ ID NO: 1 and the light chain sequence of SEQ ID NO: 2.

In one embodiment, the present invention relates to methods for the treatment of hematologic cancer in a subject who is in need thereof, comprising administering to the subject an effective amount of an anti-PD-L1 (programmed death ligand-1) antibody in combination with Compound (I), wherein Compound (I) is venetoclax or a pharmaceutically acceptable salt thereof.

In one embodiment, the present invention relates to methods for the treatment of hematologic cancer in a subject who is in need thereof, comprising administering to the subject an effective amount of an anti-PD-L1 (programmed death ligand-1) antibody in combination with Compound (I), wherein Compound (I) is venetoclax or a pharmaceutically acceptable salt thereof, and wherein the hematologic cancer is acute lymphoblastic leukemia (ALL), acute myeloid leukemia (AML), chronic myeloid leukemia (CML), chronic lymphocytic leukemia (CLL), small lymphocytic lymphoma (SLL), acute monocytic leukemia (AMoL), Hodgkin's lymphoma, non-Hodgkin lymphomas (NHL), multiple myeloma, myelodysplastic syndrome (MDS), or metastatic cutaneous squamous cell carcinoma, or locally advanced cutaneous squamous-cell carcinoma.

In one embodiment, the present invention relates to methods for the treatment of hematologic cancer in a subject who is in need thereof, comprising administering to the subject an effective amount of an anti-PD-L1 (programmed death ligand-1) antibody in combination with Compound (I), wherein Compound (I) is venetoclax or a pharmaceutically acceptable salt thereof, and wherein the anti-PD-L1 antibody is atezolizumab, avelumab, or durvalumab.

In one embodiment, the present invention relates to methods for the treatment of hematologic cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, wherein the anti-PD-L1 antibody is comprised of the heavy chain sequence of SEQ ID NO: 11 and the light chain sequence of SEQ ID NO: 12.

In one embodiment, the present invention relates to methods for the treatment of hematologic cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, wherein the anti-PD-L1 antibody is comprised of the heavy chain sequence of SEQ ID NO: 13 and the light chain sequence of SEQ ID NO: 14.

In one embodiment, the present invention relates to methods for the treatment of hematologic cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, wherein the anti-PD-L1 antibody is comprised of the heavy chain sequence of SEQ ID NO: 15 and the light chain sequence of SEQ ID NO: 16.

In one embodiment, the present invention relates to a method for the treatment of solid tumor cancer in a subject who is in need thereof, comprising administering to the subject an effective amount of either an anti-PD-1 (programmed cell death protein-1) antibody or an anti-PD-L1 (programmed death ligand-1) antibody in combination with a selective BCL-2 inhibitor or pharmaceutically acceptable salt thereof.

In one embodiment, the present invention relates to a method for the treatment of solid tumor cancer in a subject who is in need thereof, comprising administering to the subject an effective amount of either an anti-PD-1 (programmed cell death protein-1) antibody or an anti-PD-L1 (programmed death ligand-1) antibody in combination with an effective amount of an effective amount of Compound (I), Compound (II), Compound (III) or Compound (IV) or a pharmaceutically acceptable salt thereof.

In one embodiment, the present invention relates to a method for the treatment of solid tumor cancer in a subject who is in need thereof, comprising administering to the subject an effective amount of either an anti-PD-1 (programmed cell death protein-1) antibody or an anti-PD-L1 (programmed death ligand-1) antibody in combination with Compound (I), wherein Compound (I) is venetoclax or a pharmaceutically acceptable salt thereof.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the solid tumor cancer is non-small cell lung cancer, gastric cancer, melanoma, microsatellite instability-high cancer, head and neck squamous cell cancer, metastatic cutaneous squamous cell carcinoma, locally advanced cutaneous squamous-cell carcinoma, urothelial bladder cancer, colorectal cancer, liver cancer, renal cell carcinoma, breast cancer, cervical cancer or Merkel cell carcinoma.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is pembrolizumab, nivolumab, cemiplimab, or ABBV-181.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the heavy chain sequence of SEQ ID NO: 7 and the light chain sequence of SEQ ID NO: 8.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the heavy chain sequence of SEQ ID NO: 9 and the light chain sequence of SEQ ID NO: 10.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the 2 heavy chain sequence of SEQ ID NO: 54 and the light chain sequence of SEQ ID NO: 55.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the 2 heavy chain sequence of SEQ ID NO: 1 and the light chain sequence of SEQ ID NO: 2.

In one embodiment, the present invention relates to a method for the treatment of solid tumor cancer in a subject who is in need thereof, comprising administering to the subject an effective amount of an anti-PD-L1 (programmed death ligand-1) antibody in combination with Compound (I), wherein Compound (I) is venetoclax or a pharmaceutically acceptable salt thereof.

In one embodiment, the present invention relates to a method for the treatment of solid tumor cancer in a subject who is in need thereof, comprising administering to the subject an effective amount of an anti-PD-L1 (programmed death ligand-1) antibody in combination with Compound (I), wherein Compound (I) is venetoclax or a pharmaceutically acceptable salt thereof, wherein the solid tumor cancer is non-small cell lung cancer, gastric cancer, melanoma, microsatellite instability-high cancer, head and neck squamous cell cancer, metastatic cutaneous squamous cell carcinoma, locally advanced cutaneous squamous-cell carcinoma, urothelial bladder cancer, colorectal cancer, liver cancer, renal cell carcinoma, breast cancer, cervical cancer or Merkel cell carcinoma.

In one embodiment, the present invention relates to a method for the treatment of solid tumor cancer in a subject who is in need thereof, comprising administering to the subject an effective amount of an anti-PD-L1 (programmed death ligand-1) antibody in combination with venetoclax, wherein the anti-PD-L1 antibody is atezolizumab, avelumab, or durvalumab.

In one embodiment, the present invention relates to a method for the treatment of solid tumor cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, and wherein the anti-PD-L1 antibody is comprised of the heavy chain sequence of SEQ ID NO: 11 and the light chain sequence of SEQ ID NO: 12.

In one embodiment, the present invention relates to a method for the treatment of solid tumor cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, and wherein the anti-PD-L1 antibody is comprised of the heavy chain sequence of SEQ ID NO: 13 and the light chain sequence of SEQ ID NO: 14.

In one embodiment, the present invention relates to a method for the treatment of solid tumor cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, and wherein the anti-PD-L1 antibody is comprised of the heavy chain sequence of SEQ ID NO: 15 and the light chain sequence of SEQ ID NO: 16.

Anti-PD-1 antibodies and anti-PD-L1 antibodies generally comprise a heavy chain comprising a variable region (V_{H}) having three complementarity determining regions (“CDRs”) referred to herein (in N→C order) as V_{H} CDR1, V_{H} CDR2, and V_{H} CDR3, and a light chain comprising a variable region (V_{L}) having three complementarity determining regions referred to herein (in N→C order) as V_{L} CDR1, V_{L} CDR2, and V_{L} CDR3. The amino acid sequences of exemplary CDRs of exemplary anti-PD-1 antibodies and anti-PD-L1 antibodies are provided herein.

Specific exemplary embodiments of anti-PD-1 antibodies with the above CDRs are described herein. In some embodiments, an anti-PD-1 antibody has the CDRs of SEQ ID NOS: 17, 18, 19, 20, 21, 22, 23, 24, 25, 26, 27, 28, 29, 30, 31, 32, 33 and 34. In some embodiments, an anti-PD-L1 antibody has the CDRs of SEQ ID NOS: 35, 36, 37, 38, 39, 40, 41, 42, 43, 44, 45, 46, 47, 48, 49, 50, 51, 52, 56, 57, 58, 59, 60 and 61.

In one embodiment, the present invention relates to pembrolizumab, nivolumab, cemiplimab, or ABBV-181, antibodies with specificity for PD-1 (programmed cell death protein-1). Pembrolizumab is comprised of the V_{H} CDR1, V_{H} CDR2, and V_{H} CDR3 of SEQ ID NOS: 23, 24, and 25 and the V_{L} CDR1, V_{L} CDR2, and V_{L} CDR3 of SEQ ID NOS: 26, 27, and 28. Nivolumab is comprised of the V_{H} CDR1, V_{H} CDR2, and V_{H} CDR3 of SEQ ID NOS: 29, 30, and 31 and the V_{L} CDR1, V_{L} CDR2, and V_{L} CDR3 of SEQ ID NOS: 32, 33, and 34. ABBV-181 is comprised of the V_{H} CDR1, V_{H} CDR2, and V_{H} CDR3 of SEQ ID NOS: 17, 18, and 19 and the V_{L} CDR1, V_{L} CDR2, and V_{L} CDR3 of SEQ ID NOS: 20, 21, and 22. Cemiplimab is comprised of the V_{H} CDR1, V_{H} CDR2, and V_{H} CDR3 of SEQ ID NOS: 56, 57, and 58 and the V_{L} CDR1, V_{L} CDR2, and V_{L} CDR3 of SEQ ID NOS: 59, 60, and 61.

In one embodiment, the present invention relates to atezolizumab, avelumab, or durvalumab, antibodies with specificity for PD-L1 (programmed death ligand-1). Atezolizumab is comprised of the V_{H} CDR1, V_{H} CDR2, and V_{H} CDR3 of SEQ ID NOS: 35, 36, and 37 and the V_{L} CDR1, V_{L} CDR2, and V_{L} CDR3 of SEQ ID NOS: 38, 39, and 40. Avelumab is comprised of the V_{H} CDR1, V_{H} CDR2, and V_{H} CDR3 of SEQ ID NOS: 41, 42, and 43 and the V_{L} CDR1, V_{L} CDR2, and V_{L} CDR3 of SEQ ID NOS: 44, 45, and 46. Durvalumab is comprised of the V_{H} CDR1, V_{H} CDR2, and V_{H} CDR3 of SEQ ID NOS: 47, 48, and 49 and the V_{L} CDR1, V_{L} CDR2, and V_{L} CDR3 of SEQ ID NOS: 50, 51, and 52.

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the V_{H} CDR1, V_{H} CDR2, and V_{H} CDR3 of SEQ ID NOS: 23, 24, and 25 and the V_{L} CDR1, V_{L} CDR2, and V_{L} CDR3 of SEQ ID NOS: 26, 27, and 28.

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the V_{H} CDR1, V_{H} CDR2, and V_{H} CDR3 of SEQ ID NOS: 29, 30, and 31 and the V_{L} CDR1, V_{L} CDR2, and V_{L} CDR3 of SEQ ID NOS: 32, 33, and 34.

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the V_{H} CDR1, V_{H} CDR2, and V_{H} CDR3 of SEQ ID NOS: 17, 18, and 19 and the V_{L} CDR1, V_{L} CDR2, and V_{L} CDR3 of SEQ ID NOS: 20, 21, and 22.

In one embodiment, the present invention relates to methods for the treatment of hematologic cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the V_{H} CDR1, V_{H} CDR2, and V_{H} CDR3 of SEQ ID NOS: 56, 57, and 58 and the V_{L} CDR1, V_{L} CDR2, and V_{L} CDR3 of SEQ ID NOS: 59, 60, and 61.

In one embodiment, the present invention relates to methods for the treatment of hematologic cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, wherein the anti-PD-L1 antibody is comprised of the V_{H} CDR1, V_{H} CDR2, and V_{H} CDR3 of SEQ ID NOS: 35, 36, and 37 and the V_{L} CDR1, V_{L} CDR2, and V_{L} CDR3 of SEQ ID NOS: 38, 39, and 40.

In one embodiment, the present invention relates to methods for the treatment of hematologic cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, wherein the anti-PD-L1 antibody is comprised of the V_{H} CDR1, V_{H} CDR2, and V_{H} CDR3 of SEQ ID NOS: 41, 42, and 43 and the V_{L} CDR1, V_{L} CDR2, and V_{L} CDR3 of SEQ ID NOS: 44, 45, and 46.

In one embodiment, the present invention relates to methods for the treatment of hematologic cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, wherein the anti-PD-L1 antibody is comprised of the V_{H} CDR1, V_{H} CDR2, and V_{H} CDR3 of SEQ ID NOS: 47, 48, and 49 and the V_{L} CDR1, V_{L} CDR2, and V_{L} CDR3 of SEQ ID NOS: 50, 51, and 52.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the V_{H} CDR1, V_{H} CDR2, and V_{H} CDR3 of SEQ ID NOS: 23, 24, and 25 and the V_{L} CDR1, V_{L} CDR2, and V_{L} CDR3 of SEQ ID NOS: 26, 27, and 28.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the V_{H} CDR1, V_{H} CDR2, and V_{H} CDR3 of SEQ ID NOS: 29, 30, and 31 and the V_{L} CDR1, V_{L} CDR2, and V_{L} CDR3 of SEQ ID NOS: 32, 33, and 34.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the V_{H} CDR1, V_{H} CDR2, and V_{H} CDR3 of SEQ ID NOS: 17, 18, and 19 and the V_{L} CDR1, V_{L} CDR2, and V_{L} CDR3 of SEQ ID NOS: 20, 21, and 22.

In one embodiment, the present invention relates to a method for the treatment of solid tumor cancer in a subject who is in need thereof, comprising administering to the subject an effective amount of an anti-PD-1 (programmed cell death protein-1) antibody in combination with Compound (I), wherein Compound (I) is venetoclax or a pharmaceutically acceptable salt thereof, wherein the anti-PD-1 antibody is comprised of the V_{H} CDR1, V_{H} CDR2, and V_{H} CDR3 of SEQ ID NOS: 56, 57, and 58 and the V_{L} CDR1, V_{L} CDR2, and V_{L} CDR3 of SEQ ID NOS: 59, 60, and 61.

In one embodiment, the present invention relates to a method for the treatment of solid tumor cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, and wherein the anti-PD-L1 antibody is comprised of the V_{H} CDR1, V_{H} CDR2, and V_{H} CDR3 of SEQ ID NOS: 35, 36, and 37 and the V_{L} CDR1, V_{L} CDR2, and V_{L} CDR3 of SEQ ID NOS: 38, 39, and 40.

In one embodiment, the present invention relates to a method for the treatment of solid tumor cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, and wherein the anti-PD-L1 antibody is comprised of the V_{H} CDR1, V_{H} CDR2, and V_{H} CDR3 of SEQ ID NOS: 41, 42, and 43 and the V_{L} CDR1, V_{L} CDR2, and V_{L} CDR3 of SEQ ID NOS: 44, 45, and 46.

In one embodiment, the present invention relates to a method for the treatment of solid tumor cancer in a subject who is in need thereof, comprising administering to the subject an effective amount of an a anti-PD-L1 (programmed death ligand-1) antibody in combination with Compound (I), wherein Compound (I) is venetoclax or a pharmaceutically acceptable salt thereof, and wherein the anti-PD-L1 antibody is comprised of the V_{H} CDR1, V_{H} CDR2, and V_{H} CDR3 of SEQ ID NOS: 47, 48, and 49 and the V_{L} CDR1, V_{L} CDR2, and V_{L} CDR3 of SEQ ID NOS: 50, 51, and 52.

So that the disclosure may be more readily understood, select terms are defined below.

### Definitions

The terms “treat”, “treating” and “treatment” refer to a method of alleviating or abrogating a disease and/or its attendant symptoms.

The term “subject” is defined herein to include animals such as mammals, including, but not limited to, primates. In preferred embodiments, the subject is a human.

The terms “patient” and “subject” are used herein interchangeably.

“Effective amount” refers to the amount sufficient to induce a desired biological, pharmacological, or therapeutic outcome in a subject. A therapeutically effective amount means a sufficient amount of an anti-PD-1 (programmed cell death protein-1) or anti-PD-L1 (programmed death ligand-1) in combination with a selective BCL-2 inhibitor, e.g. Compound (I), to treat or prevent hematologic cancer or solid tumor cancer at a reasonable benefit/risk ratio applicable to any medical treatment.

The term “antibody” refers to an immunoglobulin (Ig) molecule, which is generally comprised of four polypeptide chains, two heavy (H) chains and two light (L) chains, or derivative thereof, that retains the epitope binding features of an Ig molecule. In an embodiment of a full-length antibody, each heavy chain is comprised of a heavy chain variable region (V_{H}) and a heavy chain constant region (CH). The CH is comprised of three domains, CH1, CH2 and CH3. Each light chain is comprised of a light chain variable region (V_{L}) and a light chain constant region (CL). The CL is comprised of a single CL domain. The V_{H} and V_{L} can be further subdivided into regions of hypervariability, termed complementarity determining regions (CDRs), interspersed with regions that are more conserved, termed framework regions (FRs). Generally, each V_{H} and V_{L} is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass.

The term “humanized antibody” refers to an antibody from a non-human species that has been altered to be more “human-like”, i.e., more similar to human germline sequences. One type of humanized antibody is a CDR-grafted antibody, in which non-human CDR sequences are introduced into human V_{H} and V_{L} sequences to replace the corresponding human CDR sequences. A “humanized antibody” is also an antibody or a variant, derivative, analog or fragment thereof that comprises framework region (FR) sequences having substantially identity (e.g., at least 80%, at least 85%, at least 90%, at least 95%, at least 98% or at least 99% identity) to the amino acid sequence of a human antibody FR sequences and at least one CDR having substantial identity (e.g., at least 80%, at least 85%, at least 90%, at least 95%, at least 98% or at least 99% identity) to the amino acid sequence of a non-human CDR. A humanized antibody may comprise substantially all of at least one, and typically two, variable domains (Fab, Fab′, F(ab′) 2, FabC, Fv) in which the sequence of all or substantially all of the CDR regions correspond to those of a non-human immunoglobulin (i.e., donor antibody) and the sequence of all or substantially all of the FR regions are those of a human immunoglobulin. The humanized antibody can also include the CH1, hinge, CH2, CH3, and CH4 regions of the heavy chain from a human antibody. In an embodiment, a humanized antibody also comprises at least a portion of a human immunoglobulin Fc region. In some embodiments, a humanized antibody only contains a humanized light chain. In some embodiments, a humanized antibody only contains a humanized heavy chain. In some embodiments, a humanized antibody only contains a humanized variable domain of a light chain and/or humanized variable domain of a heavy chain. In some embodiments, a humanized antibody contains a light chain as well as at least the variable domain of a heavy chain. In some embodiments, a humanized antibody contains a heavy chain as well as at least the variable domain of a light chain.

The term “biological activity” refers to any one or more biological properties of a molecule (whether present naturally as found in vivo, or provided or enabled by recombinant means). Biological properties include, but are not limited to, inhibiting tumor angiogenesis, inhibiting tumor-initiating/cancer stem cell maintenance, and inhibiting tumor cell chemoresistance.

The term “neutralizing” refers to counteracting the biological activity of an antigen when a binding protein specifically binds to the antigen. In an embodiment, a neutralizing binding protein binds to an antigen (e.g., a cytokine) and reduces its biologically activity by at least about 20%, 40%, 60%, 80%, 85% or more.

“Specificity” refers to the ability of a binding protein to selectively bind an antigen.

The term “potency” refers to the ability of a binding protein to achieve a desired effect, and is a measurement of its therapeutic efficacy.

The term “biological function” refers the specific in vitro or in vivo actions of a binding protein. Binding proteins may target several classes of antigens and achieve desired therapeutic outcomes through multiple mechanisms of action. Binding proteins may target soluble proteins, cell surface antigens, and/or extracellular protein deposits. Binding proteins may agonize, antagonize, or neutralize the activity of their targets. Binding proteins may assist in the clearance of the targets to which they bind, or may result in cytotoxicity when bound to cells. Portions of two or more antibodies may be incorporated into a multivalent format to achieve more than one distinct function in a single binding protein molecule.

A “stable” binding protein is one in which the binding protein essentially retains its physical stability, chemical stability and/or biological activity upon storage. A multivalent binding protein that is stable in vitro at various temperatures for an extended period of time is desirable.

The term “solubility” refers to the ability of a protein to remain dispersed within an aqueous solution. The solubility of a protein in an aqueous formulation depends upon the proper distribution of hydrophobic and hydrophilic amino acid residues, and therefore, solubility can correlate with the production of correctly folded proteins. A person skilled in the art may detect an increase or decrease in solubility of a binding protein using routine HPLC techniques and methods known to one skilled in the art.

The term “cytokine” refers to a protein released by one cell population that acts on another cell population as an intercellular mediator. The term “cytokine” includes proteins from natural sources or from recombinant cell culture and biologically active equivalents of the native sequence cytokines.

“Control” refers to a composition that does not comprise an analyte (“negative control”) or does comprise the analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (e.g., analytes).

The term “Fc region” defines the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (e.g., U.S. Pat. Nos. 5,648,260 and 5,624,821). The Fc region mediates several important effector functions, e.g., cytokine induction, antibody dependent cell mediated cytotoxicity (ADCC), phagocytosis, complement dependent cytotoxicity (CDC), and the half-life/clearance rate of antibody and antigen-antibody complexes. In some cases these effector functions are desirable for a therapeutic immunoglobulin but in other cases might be unnecessary or even deleterious, depending on the therapeutic objectives.

The term “antigen-binding portion” of a binding protein means one or more fragments of a binding protein (e.g., an antibody) that retain the ability to specifically bind to an antigen. The antigen-binding function of a binding protein can be performed by fragments of a full-length antibody, as well as bispecific, dual specific, or multi-specific formats. Examples of binding fragments encompassed within the term “antigen-binding portion” of an binding protein include (i) an Fab fragment, a monovalent fragment consisting of the V_{L}, V_{H}, CL and CH1 domains; (ii) an F(ab′)₂ fragment, a bivalent fragment comprising two Fab fragments linked by a disulfide bridge at the hinge region; (iii) an Fd fragment consisting of the V_{H} and CH1 domains; (iv) an Fv fragment consisting of the V_{L} and V_{H} domains of a single arm of an antibody, (v) a dAb fragment, which comprises a single variable domain; and (vi) an isolated complementarity determining region (CDR). Furthermore, although the two domains of the Fv fragment, V_{L} and V_{H}, are encoded by separate genes, they can be joined, using recombinant methods, by a synthetic linker that enables them to be made as a single protein chain in which the V_{L} and V_{H} regions pair to form monovalent molecules (known as single chain Fv (scFv). Such single chain antibodies are also intended to be encompassed within the term “antigen-binding portion” of an antibody. Other forms of single chain antibodies, such as diabodies are also encompassed. In addition, single chain antibodies also include “linear antibodies” comprising a pair of tandem Fv segments (V_{H}-CH1-V_{H}-CH1) which, together with complementary light chain polypeptides, form a pair of antigen binding regions.

The term “linker” means an amino acid residue or a polypeptide comprising two or more amino acid residues joined by peptide bonds that are used to link two polypeptides (e.g., two V_{H} or two V_{L} domains). Examples of such linker polypeptides are well known in the art (see, e.g., Holliger, P., Prospero, T., Winter, G. Proc. Natl. Acad. Sci. USA 90:6444-6448 (1993); Poljak, R. J., Structure 2:1121-1123 (1994)).

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat, E. A., Wu, T. T. Ann. NY Acad. Sci. 190:382-391 (1971) and, Kabat, E. A., Wu, T. T., Perry, H. M. Sequences of Proteins of Immunological Interest, Fifth Edition, U.S. Department of Health and Human Services, NIH Publication No. 91-3242 (1991)).

The Kabat definition is a standard for numbering the residues in an antibody and is typically used to identify CDR regions. See, e.g., Johnson, G., Wu, T. T., Nucleic Acids Res., 28: 214-8 (2000). The Chothia definition is similar to the Kabat definition, but the Chothia definition takes into account positions of certain structural loop regions. See, e.g., Chothia, C., Lesk, A. M. J. Mol. Biol., 196: 901-17 (1987); Chothia, C. et al., Nature, 342: 877-83 (1989). The AbM definition uses an integrated suite of computer programs produced by Oxford Molecular Group that model antibody structure. See, e.g., Martin, A. C. R., Cheetham, J. C., Rees, A. R., Proc Natl Acad Sci (USA), 86:9268-9272 (1989); “AbM.TM., A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala, R., Xia, Y., Huang, E., Levitt, M., Ab Initio Protein Structure Prediction Using a Combined Hierarchical Approach, Proteins: Structure, Function and Genetics (Suppl. 3) 194-198 (1999). The contact definition is based on an analysis of the available complex crystal structures. See, e.g., MacCallum, R. M., Martin, A. C. R., Thornton, J. M., J. Mol. Biol., 5:732-45 (1996). In another approach, referred to herein as the “conformational definition” of CDRs, the positions of the CDRs may be identified as the residues that make enthalpic contributions to antigen binding. See, e.g., Makabe, K., et al., Journal of Biological Chemistry, 283:1156-1166 (2008). Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

The term “CDR” means a complementarity determining region within an immunoglobulin variable region sequence. There are three CDRs for each epitope in each of the variable regions of the heavy chain and the light chain, which are designated CDR1, CDR2 and CDR3, for each of the heavy and light chain variable regions. The term “CDR set” refers to a group of three CDRs that occur in a single variable region capable of binding the antigen. The exact boundaries of these CDRs have been defined differently according to different systems. The system described by Kabat (Kabat et al. (1987) and (1991)) not only provides an unambiguous residue numbering system applicable to any variable region of an antibody, but also provides precise residue boundaries defining the three CDRs. These CDRs may be referred to as Kabat CDRs. Chothia and colleagues (Chothia, C., Lesk, A. M. J. Mol. Biol., 196: 901-17 (1987); Chothia, C. et al., Nature, 342: 877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan, E. A., Abergel, C., Tipper, J. P. FASEB J. 9:133-139 (1995) and MacCallum, R. M., Martin, A. C. R., Thornton, J. M., J. Mol. Biol., 5:732-45 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The term “epitope” means a region of an antigen that is bound by a binding protein, e.g., a region capable of specifically binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. In an embodiment, an epitope comprises the amino acid residues of a region of an antigen (or fragment thereof) known to bind to the complimentary site on the specific binding partner. An antigenic fragment can contain more than one epitope. In certain embodiments, a binding protein specifically binds an antigen when it recognizes its target antigen in a complex mixture of proteins and/or macromolecules. Binding proteins “bind to the same epitope” if the antibodies cross-compete (e.g., one prevents the other from binding to the binding protein, or inhibits the modulating effect on the other of binding to the binding protein). The methods of visualizing and modeling epitope recognition are known to one skilled in the art (US 20090311253).

The term “variant” means a polypeptide that differs from a given polypeptide in amino acid sequence by the addition (e.g., insertion), deletion, or conservative substitution of amino acids, but that retains the biological activity of the given polypeptide (e.g., a variant PD-1 antibody can compete with an anti-PD-1 antibody for binding to PD-1). A conservative substitution of an amino acid, i.e., replacing an amino acid with a different amino acid of similar properties (e.g., hydrophilicity and/or degree or distribution of charged regions) is recognized in the art as typically involving a minor change. These minor changes can be identified, in part, by considering the hydropathic index of amino acids, as understood in the art (see, e.g., Kyte, J., Doolittle, R. F., J. Mol. Biol. 157: 105-132 (1982)). In one aspect, amino acids having hydropathic indexes of ±2 are substituted. The hydrophilicity of amino acids can also be used to reveal substitutions that would result in proteins that retain biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., US Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also includes polypeptides or fragments thereof that have been differentially processed, such as by proteolysis, phosphorylation, or other post-translational modification, yet retain biological activity and/or antigen reactivity, e.g., the ability to bind to PD-1 and/or PD-L1. The term “variant” encompasses fragments of a variant unless otherwise defined. A variant may be 99%, 98%, 97%, 96%, 95%, 94%, 93%, 92%, 91%, 90%, 89%, 88%, 87%, 86%,85%, 84%, 83%, 82%, 81%, 80%, 79%, 78%, 77%, 76%, or 75% identical to the wild type sequence.

The terms “combination” and “combinations” as used herein refer to combination of an anti-PD-1 antibody or anti-PD-L1 antibody with a BCL-2 inhibitor, e.g. venetoclax; the concurrent administration of an anti-PD-1 antibody or anti-PD-L1 antibody with a BCL-2 inhibitor, e.g. venetoclax; the sequential administration of an anti-PD-1 antibody or anti-PD-L1 antibody with a BCL-2 inhibitor, e.g. venetoclax; or the sequential administration of a BCL-2 inhibitor, e.g. venetoclax with an anti-PD-1 antibody or anti-PD-L1 antibody.

Pembrolizumab is indicated for the treatment of patients with melanoma, non-small cell lung cancer, head and neck squamous cell cancer, classical Hodgkin lymphoma, urothelial carcinoma, microsatellite instability-high cancer, gastric cancer and cervical cancer. Pembrolizumab is administered at a dose of 200 mg as an intravenous infusion over 30 minutes every 3 weeks until disease progression or unacceptable toxicity for melanoma. Pembrolizumab is administered at a dose of 200 mg as an intravenous infusion over 30 minutes every 3 weeks until disease progression, unacceptable toxicity, or up to 24 months in patients without disease progression for non-small cell lung cancer, head and neck squamous cell cancer, classical Hodgkin lymphoma, urothelial carcinoma, microsatellite instability-high cancer, gastric cancer and cervical cancer.

Nivolumab is indicated for the treatment of patients with unresectable or metastatic melanoma, adjuvant treatment of melanoma, metastatic non-small cell lung cancer, renal cell carcinoma, classical Hodgkin lymphoma, squamous cell carcinoma of the head and neck, urothelial carcinoma, microsatellite instability-high or mismatch repair deficient metastatic colorectal cancer, and hepatocellular carcinoma. Nivolumab is administered as a single agent at a dose of 240 mg as an intravenous infusion over 30 minutes every 2 weeks until disease progression or unacceptable toxicity for unresectable or metastatic melanoma, non-small cell lung cancer, renal cell carcinoma, or urothelial carcinoma. Nivolumab is administered as a single agent at a dose of 240 mg as an intravenous infusion over 60 minutes every 2 weeks until disease recurrence or unacceptable toxicity for up to 1 year for adjuvant treatment of melanoma. Nivolumab is administered at a dose of 3 mg/kg as an intravenous infusion over 30 minutes every 2 weeks until disease progression or unacceptable toxicity for classical Hodgkin lymphoma or squamous cell carcinoma of the head and neck. Nivolumab is administered at a dose of 240 mg as an intravenous infusion over 60 minutes every 2 weeks until disease recurrence or unacceptable toxicity for colorectal cancer or hepatocellular carcinoma. Nivolumab is also administered at a dose of 1 mg/kg as an intravenous infusion over 30 minutes, followed by ipilimumab on the same day, every 3 weeks for 4 doses for unresectable or metastatic melanoma. Subsequently, nivolumab is administered as a single agent as previously described.

Atezolizumab is indicated for the treatment of patients with locally advanced or metastatic urothelial carcinoma and metastatic non-small cell lung cancer. Atezolizumab is administered at dose of 1200 mg as an intravenous infusion over 60 minutes every 3 weeks until disease progression or unacceptable toxicity. Subsequent doses may be delivered over 30 minutes if the first infusion is tolerated.

Avelumab is indicated for the treatment of patients with metastatic Merkel cell carcinoma and locally advanced or metastatic urothelial carcinoma. Avelumab is administered at a dose of 10 mg/kg as an intravenous infusion over 60 minutes every 2 weeks until disease progression or unacceptable toxicity.

Durvalumab is indicated for the treatment of patients with locally advanced or metastatic urothelial carcinoma. Durvalumab is administered at a dose of 10 mg/kg as an intravenous infusion over 60 minutes every 2 weeks until disease progression or unacceptable toxicity.

Venetoclax is indicated for the treatment of patients with chronic lymphocytic leukemia (CLL) or small lymphocytic lymphoma (SLL), with or without 17p deletion, who have received at least one prior therapy. In the treatment of CLL, venetoclax is administered at a recommended dose of 400 mg daily following a 5-week ramp-up schedule.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting. Any range described here will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. To the extent documents incorporated by reference contradict the disclosure contained in the specification, the specification will supersede any contradictory material.

Generally, nomenclatures used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein unless otherwise indicated. The nomenclatures used in connection with, and the laboratory procedures and techniques of, analytical chemistry, synthetic organic chemistry, and medicinal and pharmaceutical chemistry described herein are those well-known and commonly used in the art unless otherwise indicated.

Table 1 provides the full-length heavy, full-length light chain, and CDR sequences for binding proteins directed against programmed cell death protein 1 (PD-1) in ABBV-181.

TABLE 1
Full-length heavy chain, full-length light
chain, and CDR binding proteins of ABBV-
181 directed against epitopes of
programmed cell death protein 1
(PD-1). The underlined amino
acids represent the CDRs in
the full-length sequences.

| SEQ ID | | |
|---|---|---|
| NO: | Chain | Sequence |
| 1 | heavy | EIQLVQSGAEVKKPGSSVKVSCKASGYTF |
| | chain | THYGMNWVRQAPGQGLEWVGWVNTYTGEP |
| | | TYADDFKGRLTFTLDTSTSTAYMELSSLR |
| | | SEDTAVYYCTREGEGLGFGDWGQGTTVTV |
| | | SSASTKGPSVFPLAPSSKSTSGGTAALGC |
| | | LVKDYFPEPVTVSWNSGALTSGVHTFPAV |
| | | LQSSGLYSLSSVVTVPSSSLGTQTYICNV |
| | | NHKPSNTKVDKKVEPKSCDKTHTCPPCPA |
| | | PEAAGGPSVFLFPPKPKDTLMISRTPEVT |
| | | CVVVDVSHEDPEVKFNWYVDGVEVHNAKT |
| | | KPREEQYNSTYRVVSVLTVLHQDWLNGKE |
| | | YKCKVSNKALPAPIEKTISKAKGQPREPQ |
| | | VYTLPPSREEMTKNQVSLTCLVKGFYPSD |
| | | IAVEWESNGQPENNYKTTPPVLDSDGSFF |
| | | LYSKLTVDKSRWQQGNVFSCSVMHEALHN |
| | | HYTQKSLSLSPGK |
| 2 | light | DVVMTQSPLSLPVTPGEPASISCRSSQSI |
| | chain | VHSHGDTYLEWYLQKPGQSPQLLIYKVSN |
| | | RFSGVPDRFSGSGSGTDFTLKISRVEAED |
| | | VGVYYCFQGSHIPVTFGQGTKLEIKRTVA |
| | | APSVFIFPPSDEQLKSGTASVVCLLNNFY |
| | | PREAKVQWKVDNALQSGNSQESVTEQDSK |
| | | DSTYSLSSTLTLSKADYEKHKVYACEVTH |
| | | QGLSSPVTKSFNRGEC |
| 17 | V_{H} | GYTFTHYGMN |
| | CDR1 | |
| 18 | V_{H} | WVNTYTGEPTYADDFKG |
| | CDR2 | |
| 19 | V_{H} | EGEGLGFGD |
| | CDR3 | |
| 20 | V_{L} CDR1 | RSSQSIVHSHGDTYLE |
| 21 | V_{L} CDR2 | KVSNRFS |
| 22 | V_{L} CDR3 | FQGSHIPVT |

Table 2 provides the full-length heavy and full-length light chain sequences for binding proteins directed against murine programmed cell death protein 1 (PD-1) in anti mu PD1 (17D2 murinized, VH2xVL1x)[mu IgG2a/k] DANA.

TABLE 2
Full-length heavy chain and full-length light
chain binding proteins of anti mu PD1 (17D2
murinized, VH2xVL1x)[mu IgG2a/k] DANA
directed against epitopes of murine
programmed cell death protein 1
(PD-1).

| SEQ ID | | |
|---|---|---|
| NO: | Chain | Sequence |
| 3 | heavy | QVTLKESGPGILQSSQTLSLTCTFSGFSLST |
| | chain | YGMGVGWIRQPSGKGLEWLTNIWWDDDKYYN |
| | | SSLKNRLTVSKDTANNQVFLKFTSVDIADTA |
| | | TYYCGRMEWGGSFDYWGQGIMVTVSSAKTTA |
| | | PSVYPLAPVCGDTTGSSVTLGCLVKGYFPEP |
| | | VTLTWNSGSLSSGVHTFPAVLQSDLYTLSSS |
| | | VTVTSSTWPSQSITCNVAHPASSTKVDKKIE |
| | | PRGPTIKPCPPCKCPAPNLLGGPSVFIFPPK |
| | | IKDVLMISLSPIVTCVVVAVSEDDPDVQISW |
| | | FVNNVEVHTAQTQTHREDYASTLRVVSALPI |
| | | QHQDWMSGKEFKCKVNNKDLPAPIERTISKP |
| | | KGSVRAPQVYVLPPPEEEMTKKQVTLTCMVT |
| | | DFMPEDIYVEWTNNGKTELNYKNTEPVLDSD |
| | | GSYFMYSKLRVEKKNWVERNSYSCSVVHEGL |
| | | HNHHTTKSFSRTPGK |
| 4 | light | DIVMTQTPVSLSVSLGGQVSISCRSSQSLEH |
| | chain | RNGITFLSWYLQKPGQSPQLLIYKVSNRFSG |
| | | ISDRFSGRGSGTDFVLQINRVEPDDLGVYYC |
| | | GQGTHYPLTFGSGTKLEIKRADAAPTVSIFP |
| | | PSSEQLTSGGASVVCFLNNFYPKDINVKWKI |
| | | DGSERQNGVLNSWTDQDSKDSTYSMSSTLTL |
| | | TKDEYERHNSYTCEATHKTSTSPIVKSFNRN |
| | | EC |

Table 3 provides the full-length heavy and full-length light chain sequences for binding proteins directed against murine programmed death ligand-1 (PD-L1) in anti hu PD-L1 YW243.55.S70 [mu IgG2 a/k].

TABLE 3
Full-length heavy chain and full-length light
chain binding proteins of anti hu PD-L1
YW243.55.S70 [mu IgG2 a/k] directed
against epitopes of murine programmed
death ligand-1 (PD-L1).

| SEQ ID | | |
|---|---|---|
| NO: | Chain | Sequence |
| 5 | heavy | EVQLVESGGGLVQPGGSLRLSCAASGFTFSD |
| | chain | SWIHWVRQAPGKGLEWVAWISPYGGSTYYAD |
| | | SVKGRFTISADTSKNTAYLQMNSLRAEDTAV |
| | | YYCARRHYPGGFDYWGQGTLVTVSAAKTTAP |
| | | SVYPLAPVCGDTTGSSVTLGCLVKGYFPEPV |
| | | TLTWNSGSLSSGVHTFPAVLQSDLYTLSSSV |
| | | TVTSSTWPSQSITCNVAHPASSTKVDKKIEP |
| | | RGPTIKPCPPCKCPAPNLLGGPSVFIFPPKI |
| | | KDVLMISLSPIVTCVVVDVSEDDPDVQISWF |
| | | VNNVEVHTAQTQTHREDYNSTLRVVSALPIQ |
| | | HQDWMSGKEFKCKVNNKDLPAPIERTISKPK |
| | | GSVRAPQVYVLPPPEEEMTKKQVTLTCMVTD |
| | | FMPEDIYVEWTNNGKTELNYKNTEPVLDSDG |
| | | SYFMYSKLRVEKKNWVERNSYSCSVVHEGLH |
| | | NHHTTKSFSRTPGK |
| 6 | light | DIQMTQSPSSLSASVGDRVTITCRASQDVST |
| | chain | AVAWYQQKPGKAPKLLIYSASFLYSGVPSRF |
| | | SGSGSGTDFTLTISSLQPEDFATYYCQQYLY |
| | | HPATFGQGTKVEIKRADAAPTVSIFPPSSEQ |
| | | LTSGGASVVCFLNNFYPKDINVKWKIDGSER |
| | | QNGVLNSWTDQDSKDSTYSMSSTLTLTKDEY |
| | | ERHNSYTCEATHKTSTSPIVKSFNRNEC |

Table 4 provides the full-length heavy and full-length light chain sequences for binding proteins directed against programmed cell death protein 1 (PD-1) in pembrolizumab nivolumab, or cemiplimab and for binding proteins directed against programmed death ligand-1 (PD-L1) in atezolizumab, avelumab, or durvalumab.

TABLE 4
Full-length heavy chain, full-length light chain, and CDR binding
proteins of pembrolizumab, nivolumab and cemiplimab direct
against epitopes of programmed cell death protein 1 (PD-1)
and atezolizumab, avelumab, or durvalumab directed against
epitopes of programmed death ligand-1 (PD-L1). The
underlined amino acids represent the CDRs in the
full-length sequences.

| SEQ ID | | |
|---|---|---|
| NO: | Inhibitor/Chain | Sequence |
| 7 | pembrolizumab | QVQLVQSGVEVKKPGASVKVSCKASGYTFTNYYMYWVRQ |
| | heavy chain | APGQGLEWMGGINPSNGGTNFNEKFKNRVTLTTDSSTTT |
| | | AYMELKSLQFDDTAVYYCARRDYRFDMGFDYWGQGTTVT |
| | | VSSASTKGPSVFPLAPCSRSTSESTAALGCLVKDYFPEP |
| | | VTVSWNSGALTSGVHTFPAVLQSSGLYSLSSVVTVPSSS |
| | | LGTKTYTCNVDHKPSNTKVDKRVESKYGPPCPPCPAPEF |
| | | LGGPSVFLFPPKPKDTLMISRTPEVTCVVVDVSQEDPEV |
| | | QFNWYVDGVEVHNAKTKPREEQFNSTYRVVSVLTVLHQD |
| | | WLNGKEYKCKVSNKGLPSSIEKTISKAKGQPREPQVYTL |
| | | PPSQEEMTKNQVSLTCLVKGFYPSDIAVEWESNGQPENN |
| | | YKTTPPVLDSDGSFFLYSRLTVDKSRWQEGNVFSCSVMH |
| | | EALHNHYTQKSLSLSLGK |
| 8 | pembrolizumab | EIVLTQSPATLSLSPGERATLSCRASKGVSTSGYSYLHW |
| | light chain | YQQKPGQAPRLLIYLASYLESGVPARFSGSGSGTDFTLT |
| | | ISSLEPEDFAVYYCQHSRDLPLTFGGGTKVEIKRTVAAP |
| | | SVFIFPPSDEQLKSGTASVVCLLNNFYPREAKVQWKVDN |
| | | ALQSGNSQESVTEQDSKDSTYSLSSTLTLSKADYEKHKV |
| | | YACEVTHQGLSSPVTKSFNRGEC |
| 9 | nivolumab | QVQLVESGGGVVQPGRSLRLDCKASGITFSNSGMHWVRQ |
| | heavy chain | APGKGLEWVAVIWYDGSKRYYADSVKGRFTISRDNSKNT |
| | | LFLQMNSLRAEDTAVYYCATNDDYWGQGTLVTVSSASTK |
| | | GPSVFPLAPCSRSTSESTAALGCLVKDYFPEPVTVSWNS |
| | | GALTSGVHTFPAVLQSSGLYSLSSVVTVPSSSLGTKTYT |
| | | CNVDHKPSNTKVDKRVESKYGPPCPPCPAPEFLGGPSVF |
| | | LFPPKPKDTLMISRTPEVTCVVVDVSQEDPEVQFNWYVD |
| | | GVEVHNAKTKPREEQFNSTYRVVSVLTVLHQDWLNGKEY |
| | | KCKVSNKGLPSSIEKTISKAKGQPREPQVYTLPPSQEEM |
| | | TKNQVSLTCLVKGFYPSDIAVEWESNGQPENNYKTTPPV |
| | | LDSDGSFFLYSRLTVDKSRWQEGNVFSCSVMHEALHNHY |
| | | TQKSLSLSLGK |
| 10 | nivolumab | EIVLTQSPATLSLSPGERATLSCRASQSVSSYLAWYQQK |
| | light chain | PGQAPRLLIYDASNRATGIPARFSGSGSGTDFTLTISSL |
| | | EPEDFAVYYCQQSSNWPRTFGQGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 11 | atezolizumab | EVQLVESGGGLVQPGGSLRLSCAASGFTFSDSWIHWVRQ |
| | heavy chain | APGKGLEWVAWISPYGGSTYYADSVKGRFTISADTSKNT |
| | | AYLQMNSLRAEDTAVYYCARRHWPGGFDYWGQGTLVTVS |
| | | SASTKGPSVFPLAPSSKSTSGGTAALGCLVKDYFPEPVT |
| | | VSWNSGALTSGVHTFPAVLQSSGLYSLSSVVTVPSSSLG |
| | | TQTYICNVNHKPSNTKVDKKVEPKSCDKTHTCPPCPAPE |
| | | LLGGPSVFLFPPKPKDTLMISRTPEVTCVVVDVSHEDPE |
| | | VKFNWYVDGVEVHNAKTKPREEQYASTYRVVSVLTVLHQ |
| | | DWLNGKEYKCKVSNKALPAPIEKTISKAKGQPREPQVYT |
| | | LPPSREEMTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |
| 12 | atezolizumab | DIQMTQSPSSLSASVGDRVTITCRASQDVSTAVAWYQQK |
| | light chain | PGKAPKLLIYSASFLYSGVPSRFSGSGSGTDFTLTISSL |
| | | QPEDFATYYCQQYLYHPATFGQGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 13 | avelumab heavy | EVQLLESGGGLVQPGGSLRLSCAASGFTFSSYIMMWVRQ |
| | chain | APGKGLEWVSSIYPSGGITFYADTVKGRFTISRDNSKNT |
| | | LYLQMNSLRAEDTAVYYCARIKLGTVTTVDYWGQGTLVT |
| | | VSSASTKGPSVFPLAPSSKSTSGGTAALGCLVKDYFPEP |
| | | VTVSWNSGALTSGVHTFPAVLQSSGLYSLSSVVTVPSSS |
| | | LGTQTYICNVNHKPSNTKVDKKVEPKSCDKTHTCPPCPA |
| | | PELLGGPSVFLFPPKPKDTLMISRTPEVTCVVVDVSHED |
| | | PEVKFNWYVDGVEVHNAKTKPREEQYNSTYRVVSVLTVL |
| | | HQDWLNGKEYKCKVSNKALPAPIEKTISKAKGQPREPQV |
| | | YTLPPSRDELTKNQVSLTCLVKGFYPSDIAVEWESNGQP |
| | | ENNYKTTPPVLDSDGSFFLYSKLTVDKSRWQQGNVFSCS |
| | | VMHEALHNHYTQKSLSLSPGK |
| 14 | avelumab | QSALTQPASVSGSPGQSITISCTGTSSDVGGYNYVSWYQ |
| | light chain | QHPGKAPKLMIYDVSNRPSGVSNRFSGSKSGNTASLTIS |
| | | GLQAEDEADYYCSSYTSSSTRVFGTGTKVTVLGQPKANP |
| | | TVTLFPPSSEELQANKATLVCLISDFYPGAVTVAWKADG |
| | | SPVKAGVETTKPSKQSNNKYAASSYLSLTPEQWKSHRSY |
| | | SCQVTHEGSTVEKTVAPTECS |
| 15 | durvalumab | EVQLVESGGGLVQPGGSLRLSCAASGFTFSRYWMSWVRQ |
| | heavy chain | APGKGLEWVANIKQDGSEKYYVDSVKGRFTISRDNAKNS |
| | | LYLQMNSLRAEDTAVYYCAREGGWFGELAFDYWGQGTLV |
| | | TVSSASTKGPSVFPLAPSSKSTSGGTAALGCLVKDYFPE |
| | | PVTVSWNSGALTSGVHTFPAVLQSSGLYSLSSVVTVPSS |
| | | SLGTQTYICNVNHKPSNTKVDKRVEPKSCDKTHTCPPCP |
| | | APEFEGGPSVFLFPPKPKDTLMISRTPEVTCVVVDVSHE |
| | | DPEVKFNWYVDGVEVHNAKTKPREEQYNSTYRVVSVLTV |
| | | LHQDWLNGKEYKCKVSNKALPASIEKTISKAKGQPREPQ |
| | | VYTLPPSREEMTKNQVSLTCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| 16 | durvalumab | EIVLTQSPGTLSLSPGERATLSCRASQRVSSSYLAWYQQ |
| | light chain | KPGQAPRLLIYDASSRATGIPDRFSGSGSGTDFTLTISR |
| | | LEPEDFAVYYCQQYGSLPWTFGQGTKVEIKRTVAAPSVF |
| | | IFPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 54 | cemiplimab | EVQLLESGGVLVQPGGSLRLSCAASGFTFSNFGMTWVRQ |
| | heavy chain | APGKGLEWVSGISGGGRDTYFADSVKGRFTISRDNSKNT |
| | | LYLQMNSLKGEDTAVYYCVKWGNIYFDYWGQGTLVTVSS |
| | | ASTKGPSVFPLAPCSRSTSESTAALGCLVKDYFPEPVTV |
| | | SWNSGALTSGVHTFPAVLQSSGLYSLSSVVTVPSSSLGT |
| | | KTYTCNVDHKPSNTKVDKRVESKYGPPCPPCPAPEFLGG |
| | | PSVFLFPPKPKDTLMISRTPEVTCVVVDVSQEDPEVQFN |
| | | WYVDGVEVHNAKTKPREEQFNSTYRVVSVLTVLHQDWLN |
| | | GKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 55 | cemiplimab | DIQMTQSPSSLSASVGDSITITCRASLSINTFLNWYQQK |
| | light chain | PGKAPNLLIYAASSLHGGVPSRFSGSGSGTDFTLTIRTL |
| | | QPEDFATYYCQQSSNTPFTFGPGTVVDFRRTVAAPSVFI |
| | | FPPSDEQLKSGTASVVCLLNNFYPREAKVQWKVDNALQS |
| | | GNSQESVTEQDSKDSTYSLSSTLSKADYEKHKVYACEVT |
| | | HQGLSSPVTKSFNRGEC |
| 23 | pembrolizumab | NYYMY |
| | V_{H} CDR1 | |
| 24 | pembrolizumab | GINPSNGGTNFNEKFKN |
| | V_{H} CDR2 | |
| 25 | pembrolizumab | RDYRFDMGFDY |
| | V_{H} CDR3 | |
| 26 | pembrolizumab | RASKGVSTSGYSYLH |
| | V_{L} CDR1 | |
| 27 | pembrolizumab | LASYLE |
| | V_{L} CDR2 | |
| 28 | pembrolizumab | QHSRDLPLT |
| | V_{L} CDR3 | |
| 29 | nivolumab | NSGMH |
| | V_{H} CDR1 | |
| 30 | nivolumab | VIWYDGSKRYYADSVKG |
| | V_{H} CDR2 | |
| 31 | nivolumab | NDDY |
| | V_{H} CDR3 | |
| 32 | nivolumab | RASQSVSSYLA |
| | V_{L} CDR1 | |
| 33 | nivolumab | DASNRAT |
| | V_{L} CDR2 | |
| 34 | nivolumab | QQSSNWPRT |
| | V_{L} CDR3 | |
| 35 | atezolizumab | GFTFSDSWIH |
| | V_{H} CDR1 | |
| 36 | atezolizumab | AWISPYGGSTYYADSVKG |
| | V_{H} CDR2 | |
| 37 | atezolizumab | RHWPGGFDY |
| | V_{H} CDR3 | |
| 38 | atezolizumab | RASQDVSTAVA |
| | V_{L} CDR1 | |
| 39 | atezolizumab | SASFLYS |
| | V_{L} CDR2 | |
| 40 | atezolizumab | QQYLYHPAT |
| | V_{L} CDR3 | |
| 41 | avelumab | SYIMM |
| | V_{H} CDR1 | |
| 42 | avelumab | SIYPSGGITFYADTVKG |
| | V_{H} CDR2 | |
| 43 | avelumab | IKLGTVTTVDY |
| | V_{H} CDR3 | |
| 44 | avelumab | TGTSSDVGGYNYVS |
| | V_{L} CDR1 | |
| 45 | avelumab | DVSNRPS |
| | V_{L} CDR2 | |
| 46 | avelumab | SSYTSSSTRV |
| | V_{L} CDR3 | |
| 47 | durvalumab | RYWMS |
| | V_{H} CDR1 | |
| 48 | durvalumab | NIKQDGSEKYYVDSVKG |
| | V_{H} CDR2 | |
| 49 | durvalumab | EGGWFGELAFDY |
| | V_{H} CDR3 | |
| 50 | durvalumab | RASQRVSSSYLA |
| | V_{L} CDR1 | |
| 51 | durvalumab | DASSRAT |
| | V_{L} CDR2 | |
| 52 | durvalumab | QQYGSLPWT |
| | V_{L} CDR3 | |
| 56 | cemiplimab | GFTFSNFG |
| | V_{H} CDR1 | |
| 57 | cemiplimab | ISGGGRDT |
| | V_{H} CDR2 | |
| 58 | cemiplimab | VKWGNIYFDY |
| | V_{H} CDR3 | |
| 59 | cemiplimab | LSINT |
| | V_{L} CDR1 | |
| 60 | durvalumab | AAS |
| | V_{L} CDR2 | |
| 61 | cemiplimab | QQSSNTPFT |
| | V_{L} CDR3 | |

### Brief Description of the Sequence Listing

Incorporated herein by reference in its entirety is a Sequence Listing entitled, “12428USL2 Sequence Listing_ST25”, comprising SEQ ID NO: 1 through SEQ ID NO: 61, which includes the amino acid sequences disclosed herein. The Sequence Listing has been submitted herewith in ASCII text format via EFS. The Sequence Listing was first created on August 7, 2018, and is 60 KB in size.

### EXAMPLES

### Compound (I) Treatment of Solid Tumor Cell Lines

Solid tumor cell lines were obtained from American Type Culture Collection (ATCC, Manassas, Va.) or the National Cancer Institute (NCI) and cultured in humidified incubators at 37° C. (5% CO₂) in appropriate media (specified by supplier) supplemented with 10% fetal bovine serum (FBS) or 10% human serum (HS). 24 hours after plating into 96-well or 384-well tissue culture plates, venetoclax was added to cells at various concentrations (typically 0.010 to 10 μM in half-log increments) to perform concentration-response assessments of cell killing. After 1 to 5 days of culture in the presence of venetoclax, cell viability was assessed using CellTiter-Glo® reagent (Promega). IC₅₀ values were determined by non-linear regression analysis of the concentration response data (Table 5 and Table 6). Only one solid tumor cell line, NCI-H211, in Tables 5 and 6 showed sensitivity to treatment with clinically relevant concentrations of venetoclax.

Table 5 shows cell killing potency of Compound (I) against human solid tumor cells.

TABLE 5
Venetoclax Cell Killing Data in Human Cancer Cell Lines

| Cancer Type | Cell Line | IC₅₀ (μM) | Serum | Time (days) |
|---|---|---|---|---|
| Breast Cancer | DU4475 | >10 | 10% FBS | 5 |
| | MDA-MB-231 | 4.83 | 10% FBS | 3 |
| | SKBR3 | 4.42 | 10% FBS | 3 |
| | MDA-MB-468 | >10 | 10% FBS | 2 |
| Colorectal | COLO-201 | 9.78 | 10% FBS | 5 |
| | COLO-205 | >10 | 10% FBS | 5 |
| | COLO-320DM | >10 | 10% FBS | 5 |
| | DLD-1 | >10 | 10% FBS | 5 |
| | HCT-116 | >10 | 10% FBS | 5 |
| | HT29 | >10 | 10% FBS | 5 |
| | LS411N | 5.1 | 10% FBS | 5 |
| | LoVo | >10 | 10% FBS | 5 |
| | RKO | >10 | 10% FBS | 5 |
| | SW1463 | >10 | 10% FBS | 5 |
| | SW48 | >10 | 10% FBS | 5 |
| | SW480 | >10 | 10% FBS | 5 |
| | SW620 | >10 | 10% FBS | 5 |
| | SW948 | >10 | 10% FBS | 5 |
| | COLO-668 | >10 | 10% FBS | 5 |
| Lung | A549 | 3.19 | 10% FBS | 3 |
| | Calu-1 | >10 | 10% FBS | 3 |
| | Calu-6 | >10 | 10% FBS | 3 |
| | NCI-H1975 | >10 | 10% FBS | 3 |
| | NCI-H1993 | 3.83 | 10% FBS | 3 |
| | NCI-H2009 | 4.11 | 10% FBS | 3 |
| | NCI-H358 | >10 | 10% FBS | 3 |
| | NCI-H441 | >10 | 10% FBS | 3 |
| | NCI-H460 | >10 | 10% FBS | 3 |
| | NCI-H508 | 4.45 | 10% FBS | 3 |
| | NCI-H522 | >10 | 10% FBS | 3 |
| | NCI-H661 | >10 | 10% FBS | 3 |
| | NCI-H1048 | >10 | 10% FBS | 3 |
| | NCI-H146 | 4.20 | 10% HS | 2 |
| | NCI-H1963 | 4.59 | 10% FBS | 1 |
| | NCI-H209 | >10 | 10% FBS | 1 |
| | NCI-H510A | 1.97 | 10% FBS | 1 |
| | NCI-H524 | >10 | 10% FBS | 1 |
| | NCI-H526 | >10 | 10% FBS | 1 |
| | NCI-H69 | >10 | 10% FBS | 1 |
| | NCI-H847 | >10 | 10% FBS | 1 |
| | A431 | 4.35 | 10% FBS | 5 |
| | NCI-H1299 | >10 | 10% FBS | 5 |
| | NCI-H1417 | >5 | 10% HS | 2 |
| | NCI-H1836 | >5 | 10% HS | 2 |
| | NCI-H187 | 3.04 | 10% HS | 2 |
| | NCI-H211 | 0.03 | 10% HS | 2 |
| Melanoma | C32 | >10 | 10% FBS | 5 |
| | Hs 695T | >10 | 10% FBS | 5 |
| | WM-115 | 4.96 | 10% FBS | 5 |
| Prostate | 22Rv1 | >10 | 10% FBS | 2 |
| Hepatocellular | HCC827 | >10 | 10% FBS | 3 |
| Osteosarcoma | SJSA-1 | >10 | 10% FBS | 5 |
| Hypopharyngeal | FaDu | >10 | 10% FBS | 5 |

Table 6 shows cell killing potency of Compound (I) against murine solid tumor cells.

TABLE 6
Venetoclax Cell Killing Data vs. Murine Cancer Cells

| Cancer Type | Cell Line | IC₅₀ (μM) | Serum | Time (days) |
|---|---|---|---|---|
| Colorectal | CT26 | >10 | 10% FBS | 3 |
| Colorectal | MC38 | ~10 | 10% FBS | 3 |
| Breast Cancer | EMT6 | >10 | 10% FBS | 3 |
| Breast Cancer | 4T1 | >10 | 10% FBS | 3 |
| Lung Cancer | LL2 | >10 | 10% FBS | 3 |
| Melanoma | B16F10 | >10 | 10% FBS | 3 |
| Melanoma | B16F1 | >10 | 10% FBS | 3 |
| Renal | Renca | >10 | 10% FBS | 3 |
| Adenocarcinoma | | | | |

### Example 1: BH3 Mimetics and Checkpoint Inhibitor Antibodies in Syngeneic Tumor Models

All experiments were conducted in compliance with the National Institutes of Health Guide for Care and Use of Laboratory Animals guidelines in a facility accredited by the Association for the Assessment and Accreditation of Laboratory Animal Care (AAALAC). BALB/c, C57BL/6, CB6F1 (C57BL/6 bred with BALB/c) and SCID mice were obtained from Charles River (Wilmington, Mass.). SCID mice have severe combined immune deficiency (SCID) and are characterized by an absence of functional T- and B-cells.

EMT6, mouse mammary carcinoma and CT26, mouse colon carcinoma, cell lines were obtained from ATCC (Manassas, Va.). MC38, mouse colon carcinoma cell line was obtained from National Cancer Institute (NCI).

Compound (I),venetoclax, was formulated in 10% ethanol+30% PEG 400+60% Phosal® 50PG. Venetoclax was administered orally once a day for 14 days at 50 mg/kg/day. Mouse anti-PD-1 antibody (anti mu PD1 (17D2 murinized, VH2xVL1x)[mu IgG2a/k] DANA) and mouse anti-PD-L1 antibody (anti hu PD-L1 YW243.55.S70 [mu IgG2 a/k]) were formulated in 1× phosphate buffered saline. The mouse anti-PD-1 antibody (anti mu PD1 (17D2 murinized, VH2xVL1x)[mu IgG2a/k] DANA) or the mouse anti-PD-L1 antibody (anti hu PD-L1 YW243.55.S70 [mu IgG2 a/k]) were administered by intraperitoneal (IP) injection every 4 days, 3 times at 10 mg/kg/day.

1×10⁵ CT26, EMT6 or MC38 cells were subcutaneously injected with Matrigel into the flanks of BALB/c, CB6F1 or C57BL/6 mice, respectively. Seven days later, mice were randomized to treatment groups of vehicle, venetoclax, anti-PD-1/PD-L1 or the combination of venetoclax and anti-PD-1/PD-L1. Tumor volume was determined via measurements of the length (L) and width (W) of the tumor with electronic calipers and the volume was calculated according to the following equation: V=L×W2/2 using Study Director Version 2.1.11 (Studylog Systems Inc., South San Francisco). Complete response (CR) was defined as reduction in tumor volume to ≤25 mm³ over at least 3 successive measurements.

In the in vitro T-cell recall assay, a co-culture of lymphocytes with irradiated tumor cells was used to access the function of antigen specific memory response. CT26 (antigen specific response) or EMT6 (negative control cell line) cells were irradiated at 2000 rad and mixed with red blood cell-lysed splenocytes in 1:10 ratio. Cells were incubated in 96 well plates (round bottom) for 48 hours. Secreted IFNγ was measured by ELISA assay per manufacturer recommendation (Meso Scale Diagnostics; Rockville, Md.).

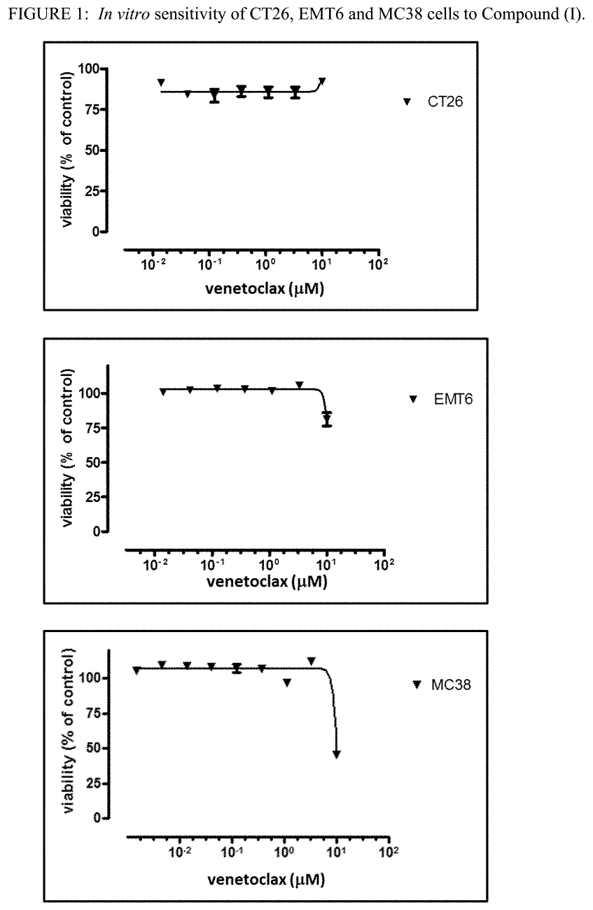

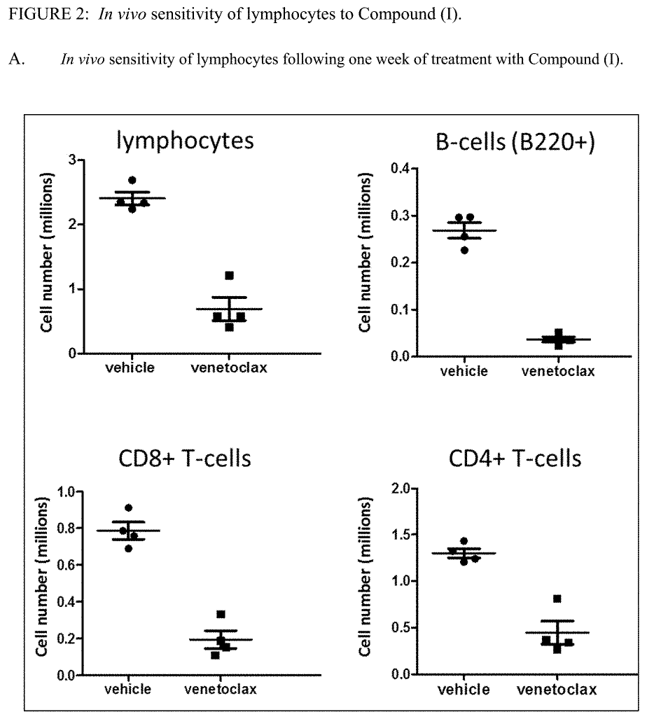

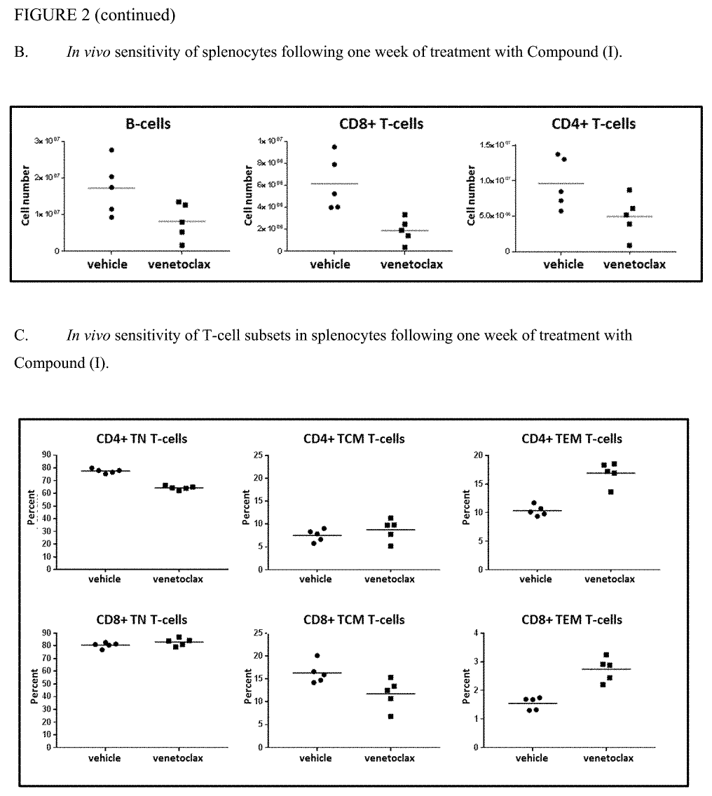

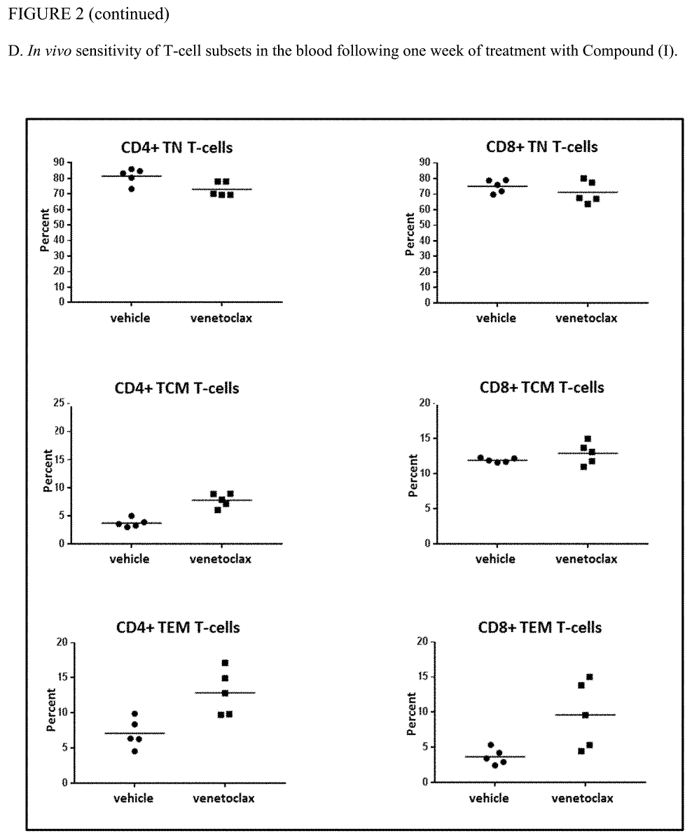

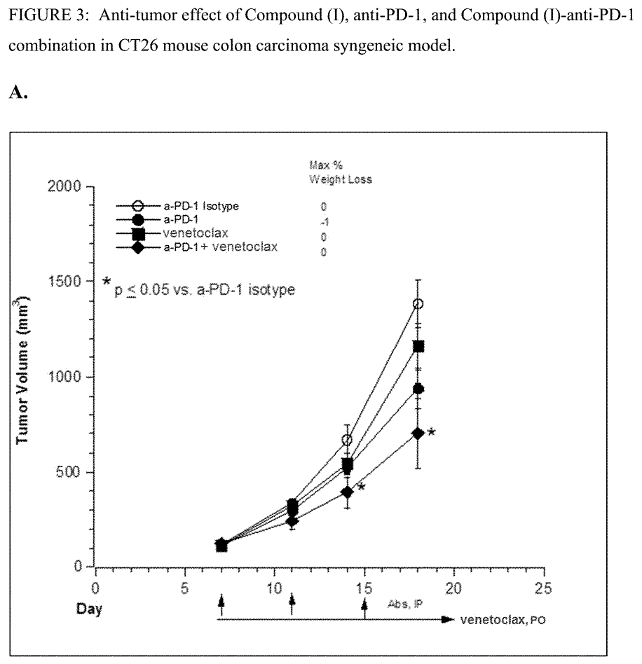

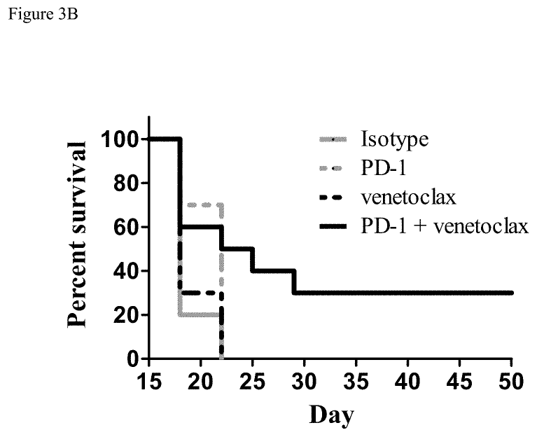

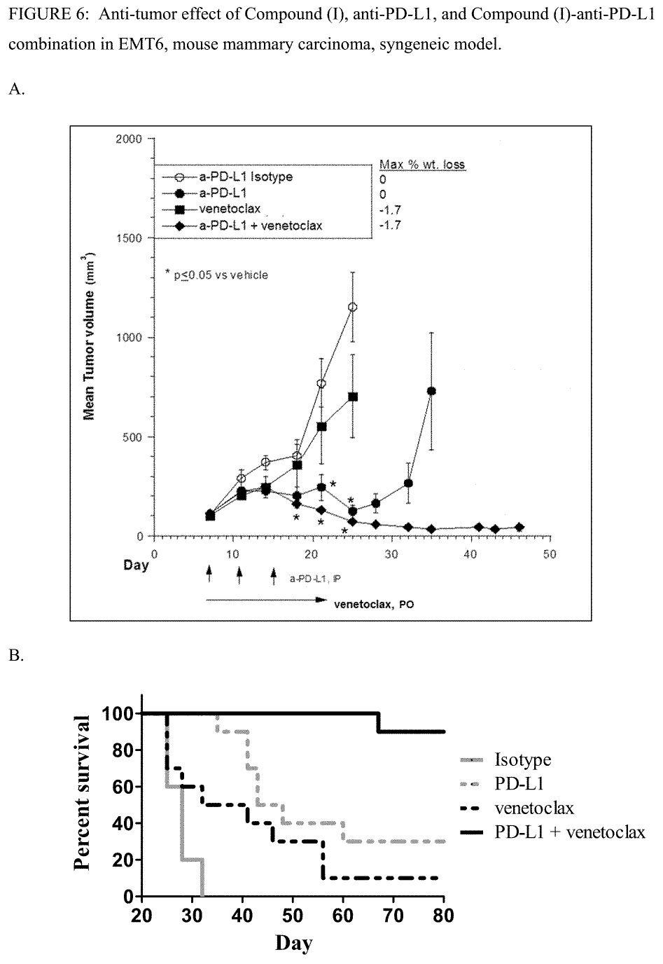

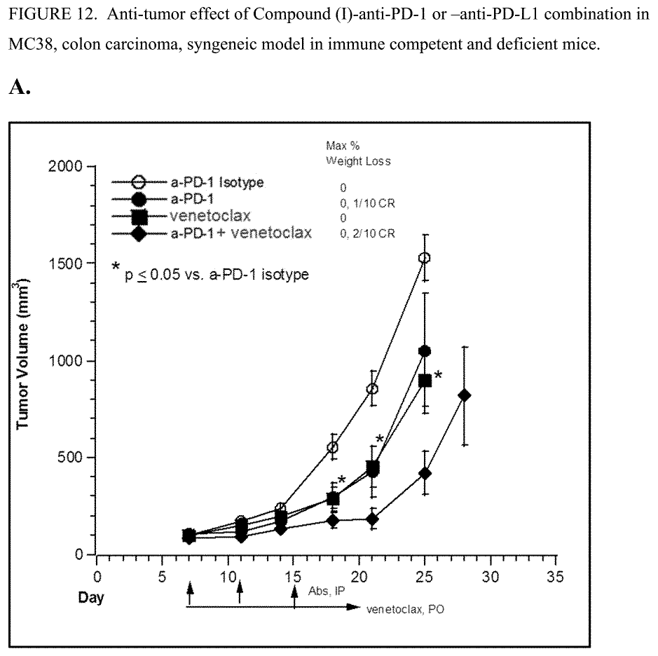

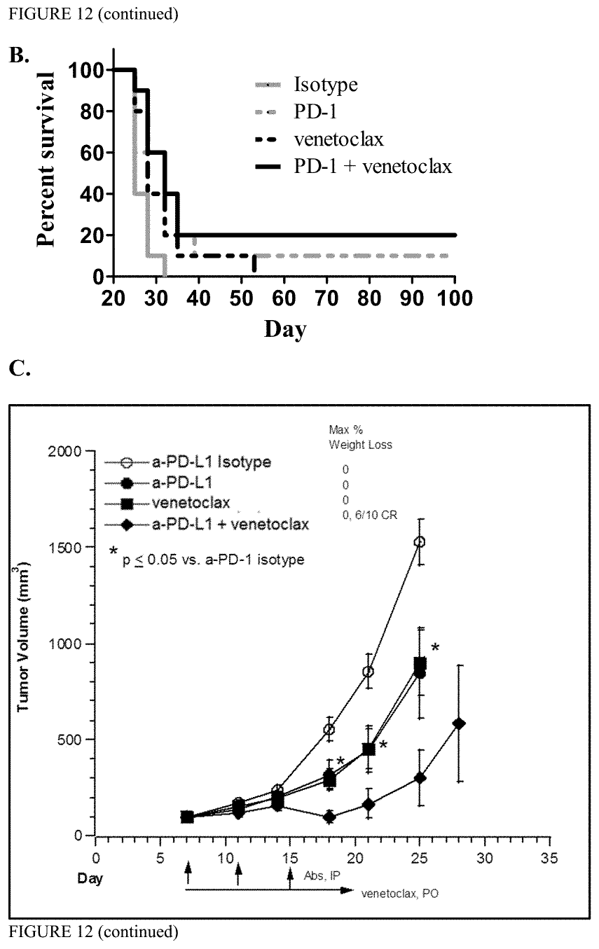

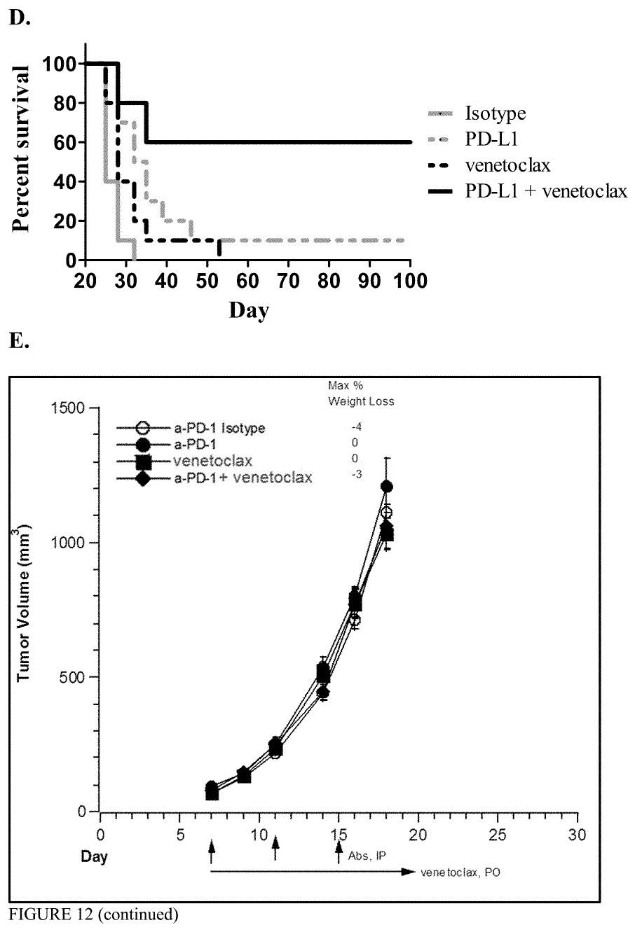

To determine whether Compound (I) would antagonize the efficacy of immune checkpoint inhibitors, venetoclax was combined with anti-PD-1 or anti-PD-L1 antibodies in a number of immunocompetent mouse models for which clear and reproducible responses to these checkpoint inhibitors have been demonstrated (Mosely, S. I., Prime, J. E., Sainson, R. C., Koopmann, J.-O., Wang, D. Y. Q., Greenawalt, D. M., Ahdesmaki, M. J., Leyland, R., Mullins, S., Pacelli, L., Marcus, D., Anderton, J., Watkins, A., Coates Ulrichsen, J., Brohawn, P., Higgs, B. W., McCourt, M., Jones, H., Harper, J. A., Morrow, M., Valge-Archer, V., Stewart, R., Dovedi, S. J., Wilkinson, R. W. Rational Selection of Syngeneic Preclinical Tumor Models for Immunotherapeutic Drug Discovery. Cancer Immunol Res. 5; 29-41 (2017)). Any reduction in checkpoint inhibitor-mediated tumor growth inhibition or response rates caused by the addition of venetoclax would indicate potential antagonism. The panel was composed of syngeneic models of solid tumors: CT26, MC38 (both colorectal carcinoma) and EMT6 (breast cancer). None of these tumor cell lines exhibited sensitivity to venetoclax-mediated apoptosis in tissue culture at clinically relevant concentrations (FIG. 1). Likewise, only modest anti-tumor activity could be observed in these models with venetoclax alone, dosed daily at 50 mg/kg for 14 days (FIG. 3, FIG. 6, FIGS. 12A, 12B, 12C, and 12D). Although venetoclax caused clear reductions in murine T-cell populations in vivo (FIG. 2A, 2B), it induced an increase in the proportion of memory cells, specifically effector memory cells (FIG. 2C, 2D), and it did not antagonize the efficacy of anti-PD-1 or anti-PD-L1 antibodies in any of the models tested. Rather, it enhanced their efficacy in certain cases.

### Example 1A: CT26 Model Treated with Compound (I)-Anti-PD-1 Antibody Combination

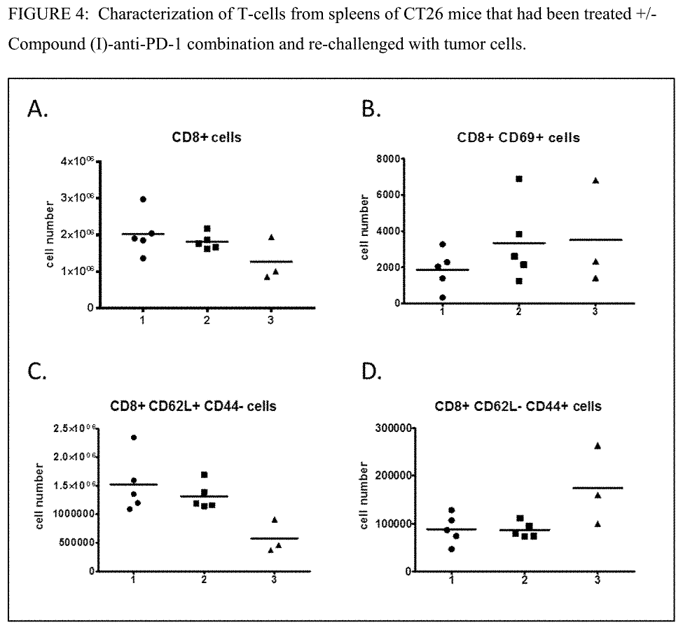

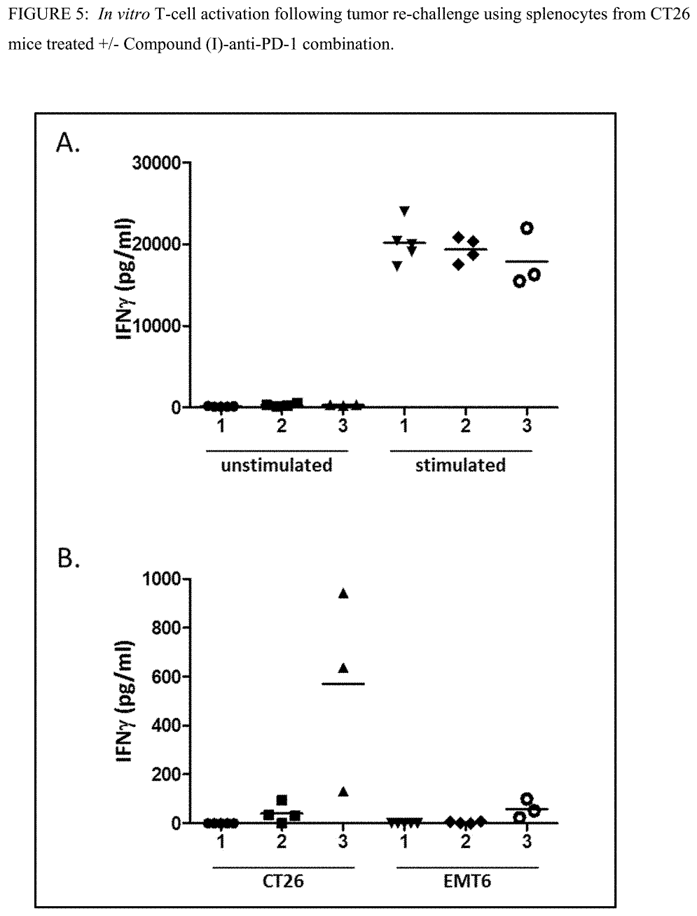

In the CT26 model, treatment with an anti-PD-1 antibody, anti mu PD1 (17D2 murinized, VH2xVL1x)[mu IgG2a/k] DANA, alone led to 32% tumor growth inhibition (TGI) and venetoclax alone led to 16% TGI whereas the anti-PD-1-venetoclax combination yielded a TGI of 49% (p<0.05 relative to control treatment with the anti-PD-1 isotype antibody on day 18) (FIG. 3A). The anti-PD-1 antibody alone did not lead to any complete responses (CR), whereas 3 of 10 mice demonstrated CR when treated with the venetoclax-anti-PD-1 combination. (FIG. 3B) All mice achieving CRs on the anti-PD-1-venetoclax combination remained tumor-free. After three months without evidence of tumor regrowth, the complete responder mice were re-inoculated with CT26 tumor cells to re-challenge the immune cells and assess anti-tumor immune memory responses. Following re-challenge, none of the mice showed any evidence of tumor engraftment, showing that these mice had mounted an immune-mediated tumor response and demonstrating that they had acquired anti-tumor immunological memory. As a control, another group of five naïve mice was also inoculated with CT26 cells at the same time and these mice, measured 10 days after inoculation, developed tumors ranging in size from 137-298 mm³. To assess the immune response of these mice, spleens were collected from three groups of mice 10 days after inoculation: (1) naïve (non-tumor bearing) BALB/c, which serve as a control cohort, (2) primary CT26 tumor-bearing mice, mentioned above, and (3) mice that had complete response to anti-PD-1-venetoclax combination and remained tumor-free when re-inoculated with CT26 cells, mentioned above. Flow cytometry analysis of splenocytes showed an increase in the number of CD8⁺ T-cells with an effector memory phenotype (CD8⁺CD62L⁻ CD44⁺) in complete responder mice that had been re-inoculated with CT26 (group 3) (FIG. 4).

To measure the ability of these splenocytes to be activated, they were cultured in the absence or presence CD3/CD28 stimulation and T-cell activation was measured by the levels of IFNγ secretion (FIG. 5A) as described in Example 1A. IFNγ levels secreted from splenocytes in group 3 were comparable to the control groups (1 and 2) indicating that venetoclax in combination with anti-PD-1 does not impair the long-term ability of T-cells to be activated. To demonstrate that there was an antigen-specific recall response, splenocytes from all groups of mice were co-cultured with irradiated CT26 or EMT6 cells. Only splenocytes from group 3 co-cultured with irradiated CT26 but not EMT6 tumor cell lines produced IFNγ (FIG. 5B), pointing to the specificity of the immune response.

The increase in the number of CD8⁺ effector memory cells and the production of IFNγ by CD8⁺ T-cells in the in vitro re-challenge response to CT26 are consistent with a recall immune response against the tumor. Importantly these data indicated that venetoclax unexpectedly enhanced the immune-mediated anti-tumor activity of the anti-PD-1 antibody and that venetoclax had no negative impact on the memory T-cell populations responsible for anti-tumor immune response.

### Example 1B: EMT6 Model Treated with Compound (I)-Anti-PD-L1 Antibody Combination

In the EMT6 model of breast cancer, venetoclax had modest activity on its own but again led to an increased number of complete responses when combined with the anti-PD-L1 antibody, anti hu PD-L1 YW243.55.S70 [mu IgG2 a/k], (9 CRs for anti-PD-L1-venetoclax versus 3 CRs for anti-PD-L1 alone) (FIG. 6). Anti-PD-L1 antibody alone led to 89% TGI and the anti-PD-L1-venetoclax combination yielded a TGI of 94% (both p<0.05 relative to vehicle on day 25).

### Example 1C: MC38 Model Treated with Compound (I)-Anti-PD-1 or Anti-PD-L1 Antibodies Combination

In the MC38 model of colon carcinoma, treatment with venetoclax, anti-PD-1 antibody, anti mu PD1 (17D2 murinized, VH2xVL1x)[mu IgG2a/k] DANA, or anti-PD-L1 antibody, anti hu PD-L1 YW243.55.S70 [mu IgG2 a/k], alone resulted in TGI of 41%, 32% and 45%, respectively whereas the anti-PD-1- or anti-PD-L1-venetoclax combinations yielded a TGI of 73% or 80% respectively (p<0.01 of the anti-PD-1-venetoclax combination compared to the vehicle control; for the combination with anti-PD-L1: p<0.0001 compared to control and p<0.01 compared to anti-PD-L1 monotherapy on day 25) (FIGS. 12A, and C).

The anti-PD-1 or anti-PD-L1 antibodies alone resulted in 1 or no mouse of 10 with any complete responses, respectively, whereas 2 of 10 and 6 of 10 mice demonstrated CR when treated with the venetoclax-anti-PD-1 or anti-PD-L1 combination, respectively. (FIGS. 12B and 12D). All mice achieving CRs remained tumor-free. After more than three months without evidence of tumor regrowth, the complete responder mice were re-inoculated with MC38 tumor cells to re-challenge the immune cells and assess anti-tumor immune memory responses. Following re-challenge, none of the mice showed any evidence of tumor engraftment, indicating that these mice had mounted an immune-mediated tumor response and demonstrating that they had acquired anti-tumor immunological memory.

To further test the direct effects of venetoclax on the immune cells, SCID mice were transplanted with MC38 cells and treated with vehicle or anti-PD-1/PD-L1/venetoclax as single agent and in combinations (FIGS. 12E and 12F). As expected, tumor growth in SCID mice treated with anti-PD-1 and anti-PD-L1 were similar to vehicle control. Similar to the anti-PD-1/PD-L1, venetoclax treatment in immunodeficient mice did not show anti-tumor efficacy, as single agent and in combination with these agents, indicating no effect on tumor cells per se and highlighting the role of venetoclax in modulating immune response in MC38 model.

### Example 2: In Vitro Sensitivity of Human Peripheral Blood Mononuclear Cells (PBMCs) or Mouse Splenocytes to Venetoclax

Human PBMCs were thawed and cultured overnight in 30 U/mL of interleukin-2 (IL-2). Cells were harvested, washed once with complete media and counted. PBMCs were plated at a density of 5×10⁵ cells per well in a 48-well plate with 30 U/mL IL-2. For stimulation of T-cells, cells were plated in anti-CD3-coated wells (2.5 μg/mL; ThermoFisher cat. #16-0037-85, clone OKT3) and soluble anti-CD28 was added (1 μg/mL; ThermoFisher cat. #16-0289-85, clone CD28.2). Cells were treated with increasing concentrations of venetoclax for 24 hours (dimethyl sulfoxide (DMSO) was used as control). Cells were harvested, stained and subjected to flow cytometry analysis with a BD LSRII instrument (BD Biosciences)

Mouse splenocytes were plated at a density of 5×10⁵ cells per well in a 48-well plate with 30 U/mL IL-2. Cells were cultured for 24 hours and then increasing concentrations of venetoclax were added for an additional 24 hours (DMSO was used as control). Cells were harvested, stained and subjected to flow cytometry analysis with an LSRFortessa™ X-20 instrument (BD Biosciences).

The syngeneic tumor studies indicated that, although Compound (I) (venetoclax) is not likely mediating a significant amount of direct tumor cell killing in these models, it contributed to enhanced efficacy in the presence of checkpoint inhibitors. To explore the potential mechanism for the enhanced efficacy, studies were conducted in vitro to further characterize T-cells, with focus on T-cell subsets, T-cell activation and T-cell function (Examples 2, 3 and 4).

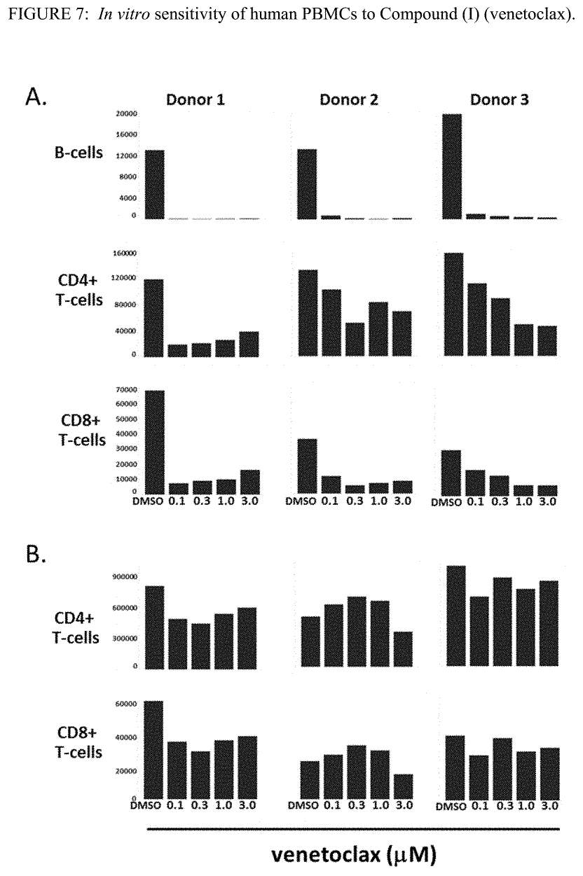

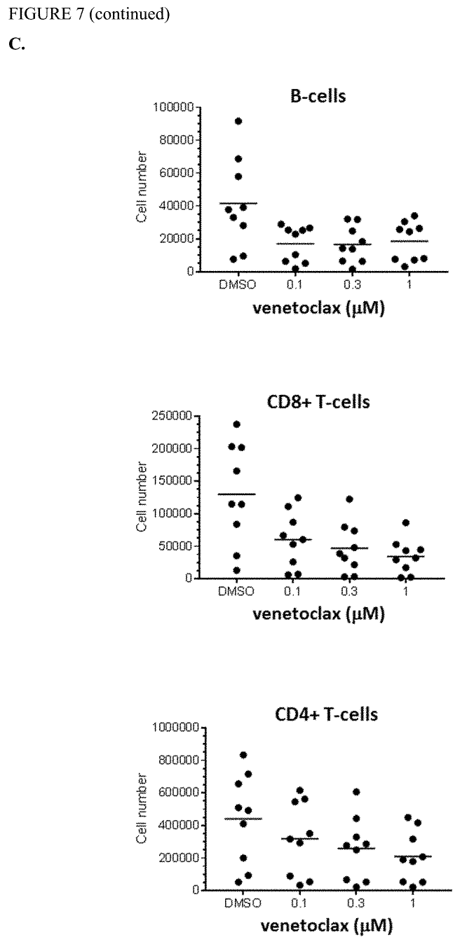

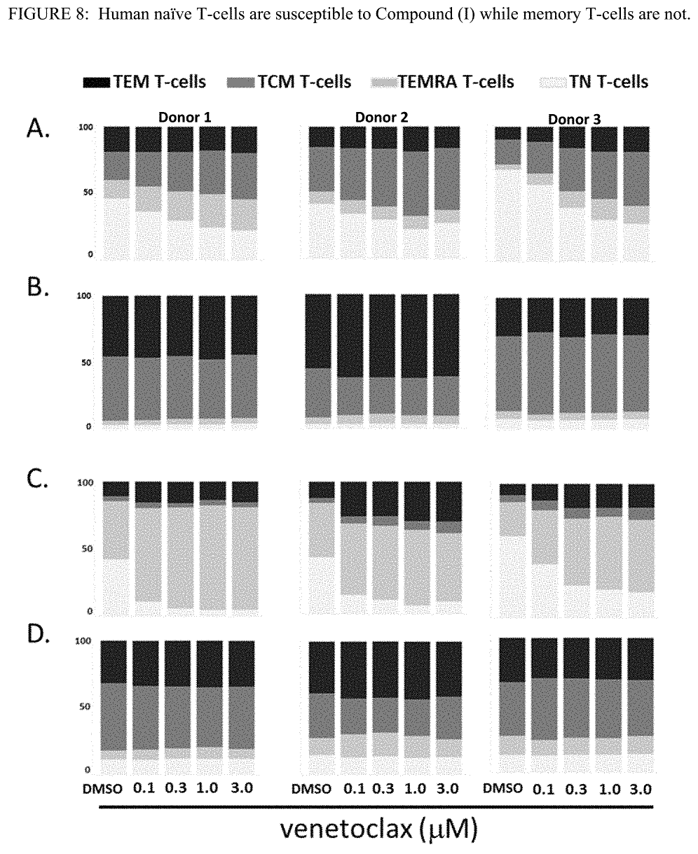

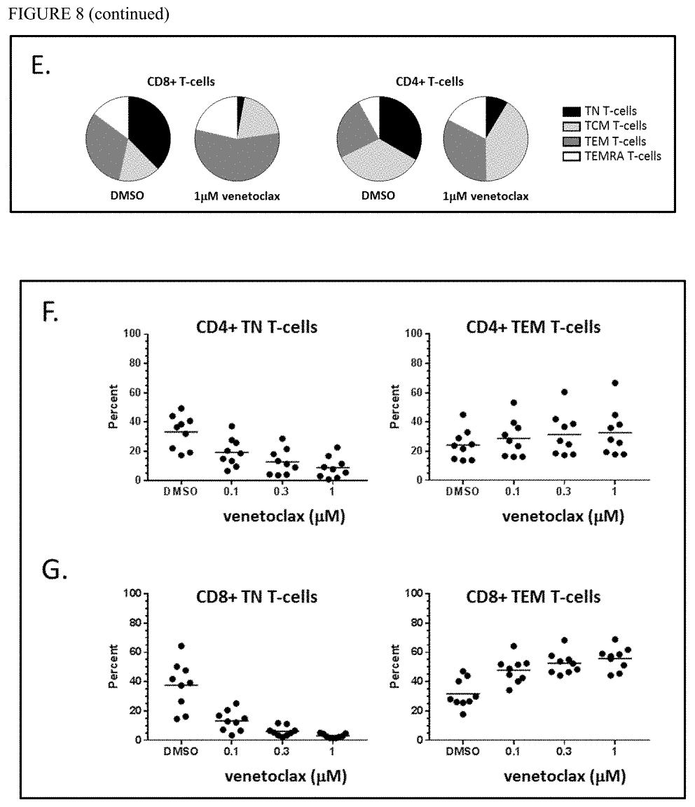

When human PBMCs were cultured for 72 hours and then treated with venetoclax (under unstimulated conditions) a dose-dependent decrease in the number of B-cells, and T-cells (CD4⁺ and CD8⁺ T-cells) was observed (FIG. 7A). However, when PBMCs were stimulated with anti-CD3 and anti-CD28 for 72 hours before treating with venetoclax, the T-cells were not sensitive to venetoclax. (FIG. 7B). While the proportion of naïve T-cells (CD4⁺CD62L⁺CD45RO⁻ and CD8⁻CD62L⁺CD45RO⁻) decreased with increasing venetoclax concentrations, the proportion of CD4⁺ central memory (TCM; CD62L⁺CD45RO⁺) and CD8⁺ terminally differentiated effector memory (TEMRA; CD8⁺CD62L⁻CD45RO⁻) and effector memory cells (TEM; CD8⁺CD62L⁻CD45RO⁺) increased relative to the number of surviving T-cells (FIGS. 8A and 8C). Under CD3/CD28 stimulated conditions, venetoclax did not affect the proportion of the T-cell subsets (FIGS. 8B and 8D). In separate experiments, human PBMCs were thawed, rested overnight and then treated with venetoclax for 24 hours. Similar results were observed when increasing the number of tested samples (n=9 donors), and refining the markers for the different T-cell subsets: naïve T-cells (TN; CD62L+CD45RA+), central memory (TCM; CD62L+CD45RA−), effector memory cells (TEM; CD62L−CD45RA−) and terminally differentiated effector memory (TEMRA; CD62L−CD45RA+) (FIGS. 8E, 8F, 8G). Venetoclax decreases the proportion of naïve T-cells, but not memory cells, and specifically induces an increase in effector memory T-cells. It can therefore be seen that, while naïve T- and B-cells were sensitive to venetoclax, memory T-cells were less sensitive to venetoclax. While TCM cells home to lymph nodes and efficiently stimulate dendritic cells, TEM and TEMRA cells home to tissues, comprised of antigen-experienced cells and are ready to mediate rapid effector and cytotoxic functions necessary to kill the tumor.

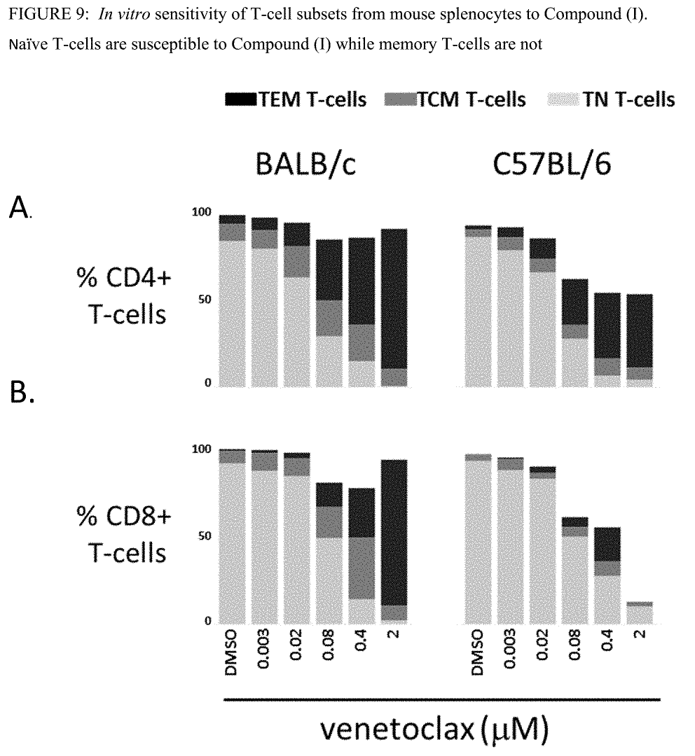

To further explore the mechanism for immune modulation in mice, splenocytes from BALB/c and C57BL/6 mice were treated in vitro with venetoclax for 24 hours. The two types of memory T-cells in mice are central memory (TCM; CD62L⁺CD44⁻) and effector memory T-cells (TEM; CD62L⁻CD44⁻). While naïve T-cells (CD62L⁺CD44⁻) were sensitive to venetoclax, effector memory T-cells were not and, importantly, their proportion increased relative to surviving T-cells with increased concentrations of venetoclax. Central memory T-cells were not affected by venetoclax under these experimental conditions (FIG. 9).

In total, memory T cells are more resistant than naïve T-cells in human PBMCs and mouse splenocytes to venetoclax treatment in vitro. Modulation of the immune system by venetoclax demonstrates its use for immune-based cancer therapy, as memory T-cells can rapidly acquire effector and cytotoxic function to eliminate cancer cells.

### Example 3: Cytomegalovirus (CMV) Recall Assay

The functional effects of Compound (I) on T-cell activation was assessed using two approaches: 1) cytomegalovirus (CMV) recall assay (Example 3), in which CMV-specific T-cells were restimulated with CMV antigen, and 2) mixed lymphocyte reactions (MLR; Example 4), in which monocytic-derived dendritic cells (MoDCs) from one donor were cultured with T-cells from another donor.

### Example 3A: CMV Recall Assay Using Human CMV+PBMC

The cytomegalovirus (CMV) recall assay was used to assess the functional effects of Compound (I) on antigen-specific immune cells. This assay used CMV antigen to stimulate pre-existing memory T-cells in CMV-positive human PBMCs. Human CMV-positive PBMCs and CMV antigen were purchased from Astarte Biologics (Cat.No:1001 and Cat. No. 1004, respectively). To measure whether T cells from CMV-positive donor are functionally active, 2×10⁵ CMV-positive PBMCs were stimulated with 0.05 μg/mL of CMV antigen and at the same time venetoclax was added. The cells were cultured in Roswell Park Memorial Institute (RPMI) media supplemented with 10% fetal bovine serum (FBS). On day 4, cells were quickly re-stimulated with phorbol 12-myristate-13-acetate (PMA, 0.08 μM)/ionomycin (1.3 μM) and treated with brefeldin A (5 μg/mL) for 4 hours. Cells were stained with the following commercial antibodies: CD3, CD4, IFNγ, and IL-2 (all from Biolegend), as well as with the Zombie Green™ fixable viability kit. Cells were analyzed using an LSRFortessa™ X-20 instrument (BD Biosciences).

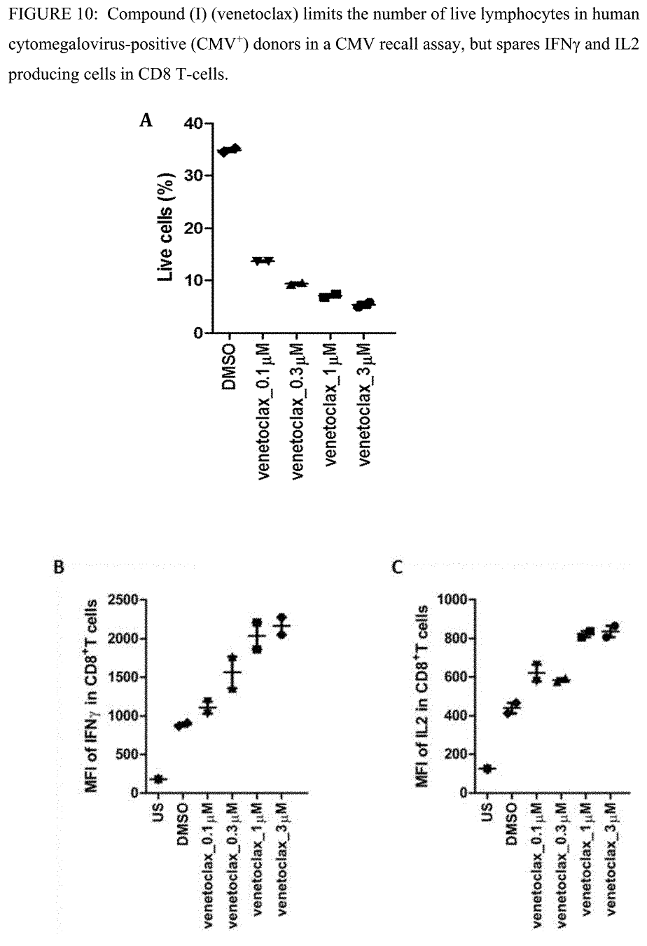

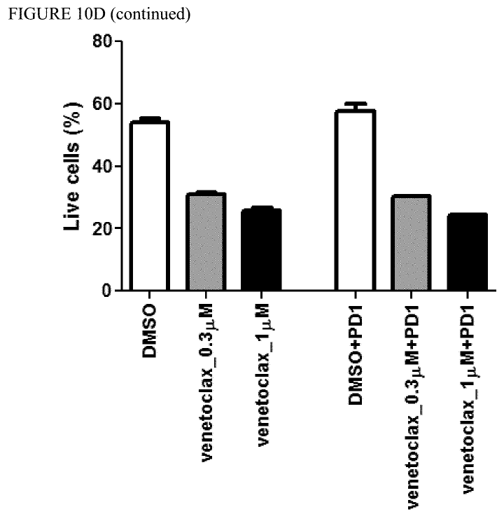

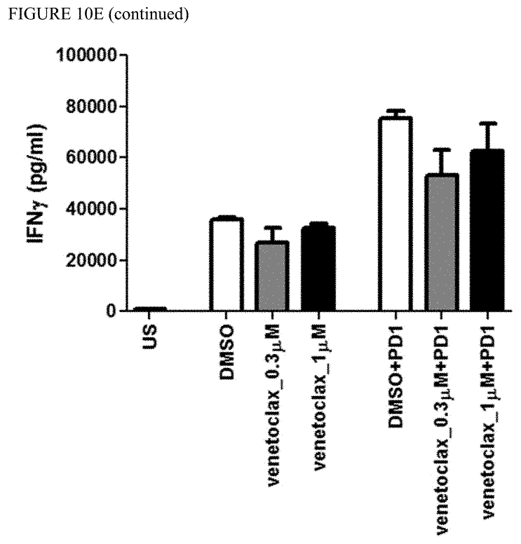

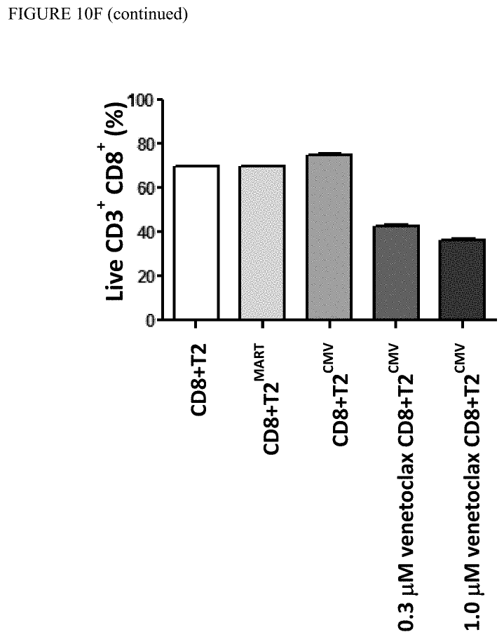

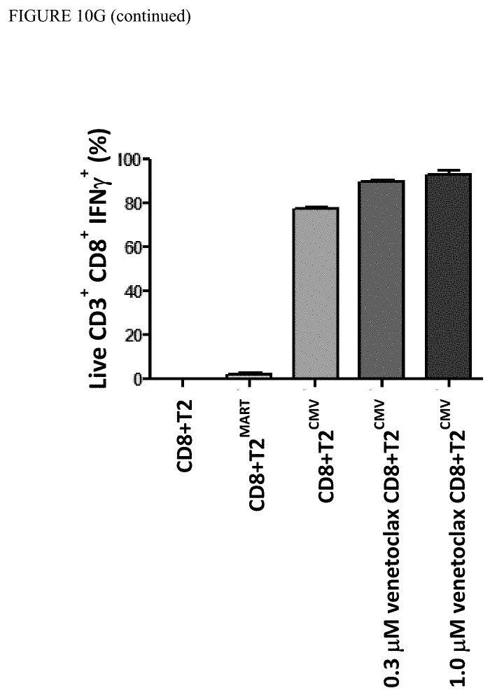

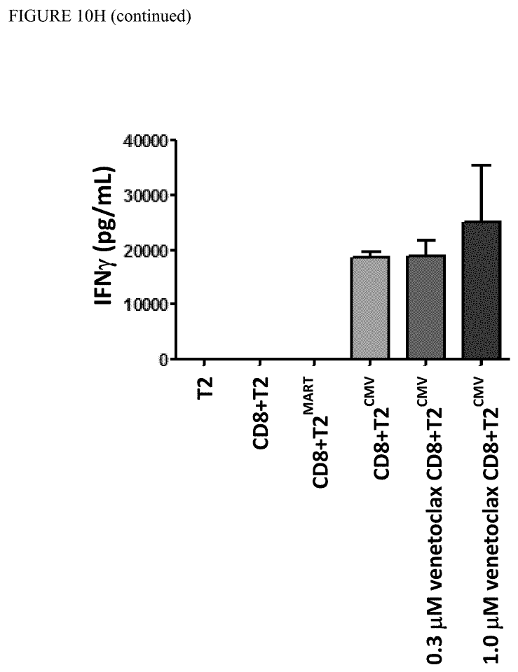

Compound (I) (venetoclax) reduced the total number of lymphocytes in a dose-dependent manner, consistent with previous examples (FIG. 10A). The remaining live cells were gated by CD3 and CD4 expression whereas CD3⁺CD4⁺ cells were considered as CD4⁺ T-cells and CD3⁺CD4⁻ cells were considered as CD8⁺ T-cells. Production of IFNγ and IL-2 cytokines by CD8+ T-cells was assessed by flow cytometry (FIG. 10B, 10C).

Although venetoclax treatment caused dose-dependent reductions in viable T-cells, a corresponding dose-dependent inversely proportional increase in remaining T-cell activation was observed as measured by mean fluorescence intensity (MFI) production of IFNγ (FIG. 10B) and IL-2 (FIG. 10C). These data indicate that venetoclax does not affect IFNγ production from antigen-specific activated T-cells on its own.

### Example 3B: CMV Recall Assay in the Presence of Anti-PD-1 Antibody

The cytomegalovirus (CMV) recall assay was used assess the functional effects of venetoclax on antigen-experienced immune cells and to evaluate the effect of venetoclax on anti-PD1 immune response. This assay used cytomegalovirus (CMV) antigen to stimulate pre-existing memory T-cells in CMV-positive human PBMCs. Human CMV-positive PBMCs (Cat.No:1001; Lot. No. 3634JY17) and CMV antigen (Cat. No. 1004) were purchased from Astarte Biologics. 2×10⁵ CMV-positive PBMCs were stimulated with 1 μg/mL of CMV antigen with or without inhibitors +/−anti-PD-1 (nivolumab) in Roswell Park Memorial Institute (RPMI) media supplemented with 10% fetal bovine serum (FBS) and 1% antibiotics. On day 4, secreted IFNγ was measured by ELISA and the live cells were analyzed using a live dead stain, Zombie Green™ (BioLegend®) using LSRFortessa™ X-20 instrument (BD Biosciences). Venetoclax reduced the percentage of live cells compared to DMSO as single agent or in combination with nivolumab (FIG. 10D). However, IFNγ secretion from antigen specific CMV+ T cells remained comparable to DMSO control and combining venetoclax with nivolumab did not affect the anti-PD-1 response (FIG. 10E).

### Example 3C: CMV Specific CD8 T Cell Response

The cytomegalovirus (CMV) specific assay was used to evaluate the effects of venetoclax on antigen-experienced CD8 T cells. This assay used CMV) peptide pp65 (NLVPMVATV, SEQ ID NO: 53) to stimulate CMV-positive CD8 T cells, and MART (ELAGIGILTV) peptide as a negative control. Human CMV-positive CD8 T cells and CMV peptide pp65 were purchased from Astarte Biologics and MART peptide as purchased from Genscript. 2×10⁵ CMV-positive CD8 T cells were stimulated with 2×10⁵ CMV pp65 peptide or MART peptide loaded T2 cells (2.5 μg/ml of peptide) with brefeldin-A in RPMI media supplemented with 10% fetal bovine serum (FBS). For measuring the IFNγ secretion in the supernatant, CMV+ CD8 T cells were activated with CMV pp65 peptide or MART peptide loaded T2 cells and incubated with venetoclax without brefeldin-A in RPMI media supplemented with 10% fetal bovine serum (FBS). After an overnight (12-14 hours) incubation period with venetoclax, cells were stained with the following antibodies: CD3, CD8, and IFN-γ (all from BioLegend), as well as with the Zombie Green™ fixable viability kit. Cells were analyzed using an LSRFortessa™ X-20 instrument (BD Biosciences). The secreted IFNγ in supernatant was measured by ELISA.

CMV+ CD8 T cells were activated by CMV pp65 peptide (NLVPMVATV) loaded on T2 cells and the effect of venetoclax on the function of these cells was assessed by measuring intra-cellular and secreted IFNγ (flow cytometry and ELISA as described above). As controls, CMV specific CD8 T cells were incubated with T2 cells without peptide or T2 cells loaded with control peptide MART (serving as negative control). As shown in FIG. 10F, venetoclax treatment reduced the number of CD8 T cells. However, the cells that survived venetoclax treatment produced similar amount of IFNγ as DMSO control as measured by flow cytometry analysis and IFNγ secretion (FIGS. 10G and 10H, respectively). Antigen specific T cells are important for anti-tumor immune response. Recently it has been shown by Horton et al., that apoptosis of antigen specific T cells compromised anti-tumor response (Horton, B. L., Williams, J. B., Cabanov, A., Spranger, S. and Gajewski, T. F. Intratumoral CD8+ T-cell Apoptosis Is a Major Component of T-cell Dysfunction and Impedes Antitumor Immunity. Cancer Immunol Res, 6(1), 14-24 (2018)). The results show that venetoclax did not affect the function of antigen specific immune response, nor the anti-PD-1 activity on antigen specific T-cells.

### Example 4: Mixed Lymphocyte Reaction (MLR) Assay

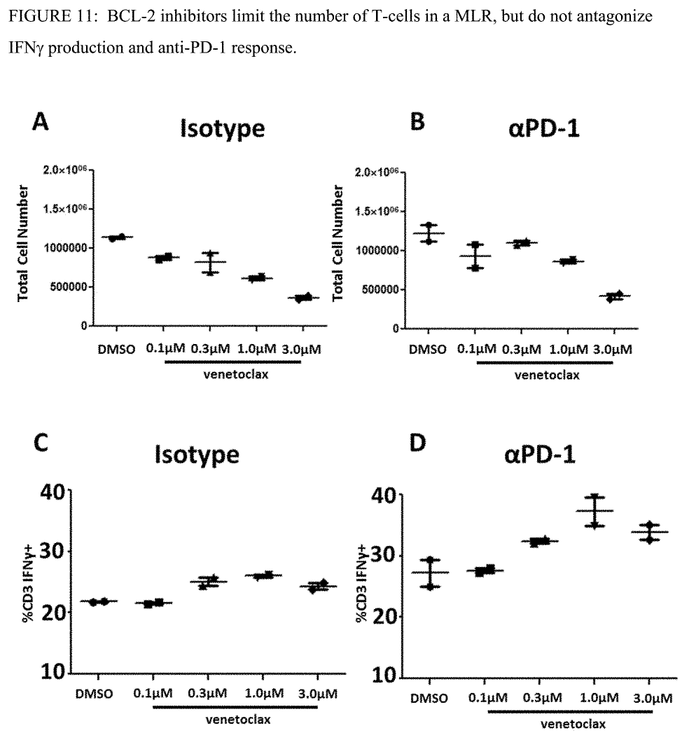

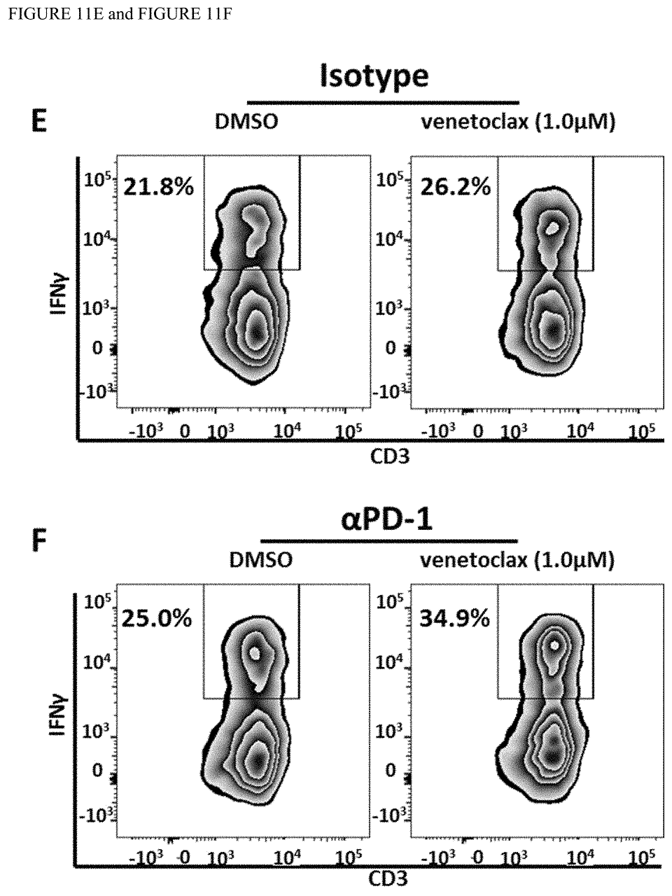

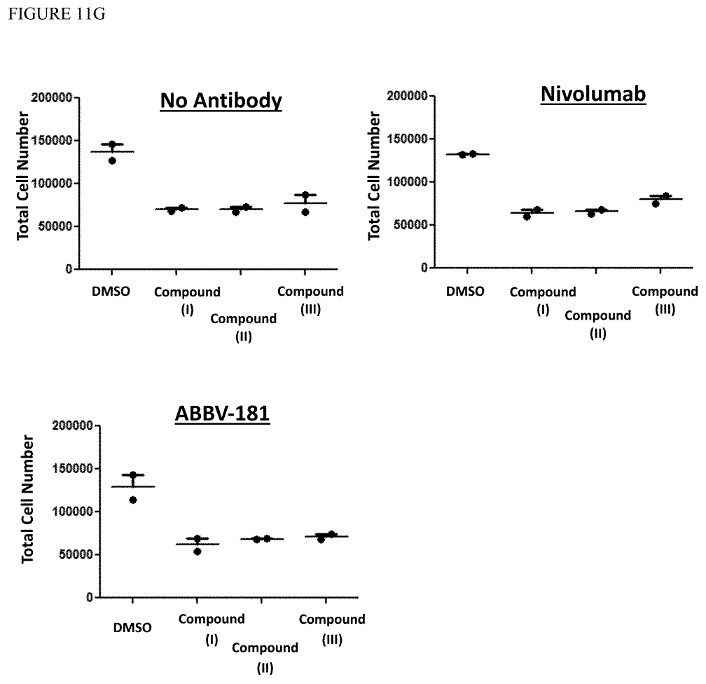

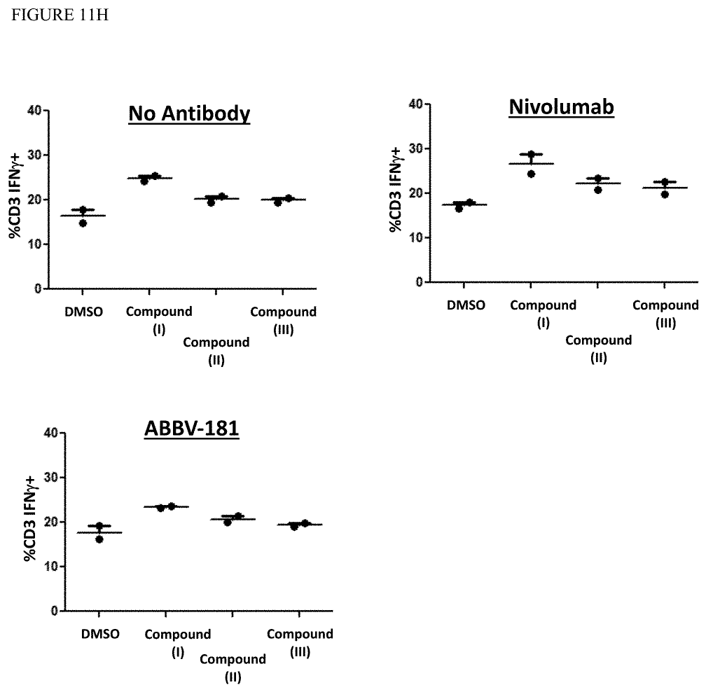

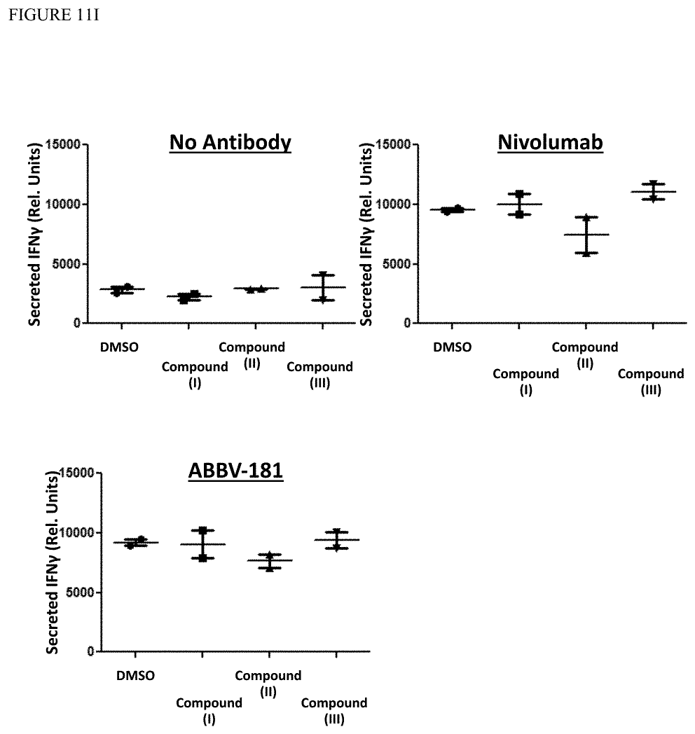

Mixed Lymphocyte Reaction (MLR) assay was used to examine venetoclax effect on T-cell function in response to immune stimulation. Monocytic-derived dendritic cells (MoDCs) were generated from fresh human blood. PBMCs were isolated using a Ficoll gradient. The PBMCs were allowed to adhere for 2 hours. After 2 hours, cells in suspension were removed. Fresh AIM V™ medium (ThermoFisher cat. #12055091) supplemented with granulocyte-macrophage colony-stimulating factor (GM-CSF, 80 ng/mL) and IL-4 (50 ng/mL) was added to the culture. After 5 days, the MoDCs were stimulated with IL-1α and TNF-α (0.2 ng/mL each) for 48 hours to increase major histocompatibility complex class II molecules (MHCII) expression. These activated MoDCs were then co-cultured with CD4 T-cells (Biospecialty Corp.) at a ratio of 10:1 (T-cells:MoDCs) in a mixed lymphocyte reaction (MLR). The cells were treated with control IgG (Isotype) or anti-PD-1 antibody (nivolumab or ABBV-181 along with either dimethyl sulfoxide (DMSO) or BCL-2 inhibitors venetoclax (Compound (I)) or Compounds (II) or (III). Antibodies were added at 10 μg/mL and the compound concentrations of venetoclax were as indicated in FIG. 11. The MLR was cultured for 5 days, after which the cells were analyzed by flow cytometry using an LSRFortessa™ X-20 instrument (BD Biosciences) to determine cell number and functional cytokine (IFNγ) responses.

As shown in the MLR experiment, the anti-PD-1 antibody, nivolumab, increased both the cell number and the proportion of CD4⁺ T-cells that produce IFNγ (FIG. 11B compared to FIG. 11A and FIG. 11D compared to FIG. 11C), as previously demonstrated (Wang, C., Thudium, K. B., Han, M., Wang, X. T., Huang, H., Feingersh, D., Garcia, C., Wu, Y., Kuhne, M., Srinivasan, M., Singh, S., Wong, S., Garner, N., Leblanc, H., Bunch, R T., Blanset, D., Selby, M. J., Korman, A. J. In Vitro Characterization of the Anti-PD-1 Antibody Nivolumab, BMS-936558, and In Vivo Toxicology in Non-Human Primates. Cancer Immunol Res. 2(9):846-56 (2014)). Venetoclax reduced the number of CD4⁺ T-cells in a dose-dependent manner (FIGS. 11A and B). However, there is an increase in the proportion of CD4⁺ T-cells that produce IFNγ when venetoclax was added with either the isotype control or the anti-PD-1 antibody (FIGS. 11C and D). Representative flow cytometry data are also shown (FIGS. 11E and 11F). Furthermore, while there are fewer CD4⁺ T-cells, there was the same amount of IFNγ detected in the supernatant (FIG. 11H).

The assay was also performed with Compound (I), Compound (II), and Compound (III) (all at 1 μM) either without an anti-PD-1-antibody or with the anti-PD-1 antibodies, nivolumab or ABBV-181. The results showed that the CD3⁺ T cell numbers declined comparably upon treatment with BCL-2 inhibitors or with any BCL-2 inhibitor in combination with an anti-PD-1 antibody (FIG. 11G). An increase in the proportion of CD3⁺ T-cells that produce IFNγ when a BCL-2 inhibitor was added alone or in combination with an anti-PD-1 antibody was observed (FIG. 11H). Again it was demonstrated that production of IFNγ was not antagonized by any of the BCL-2 inhibitors alone or in combination with any of the anti-PD-1 antibodies (FIG. 11I).

### Example 5: Effect of Venetoclax on T-cell Subsets in Human Subjects

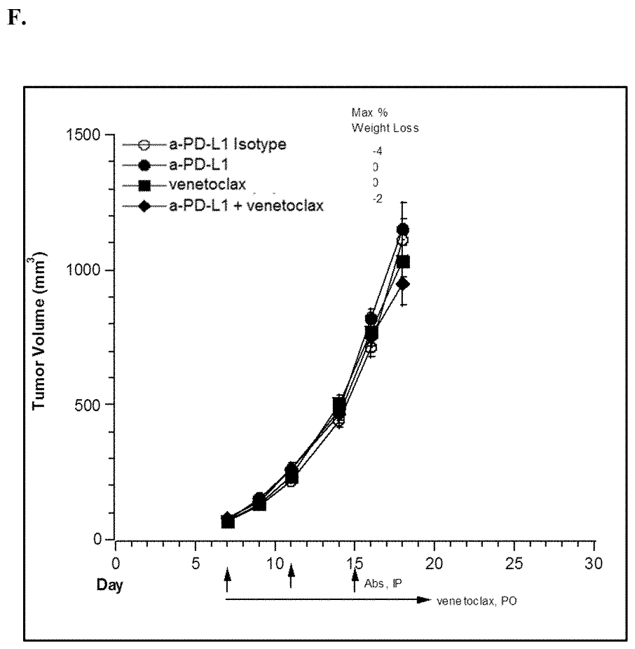

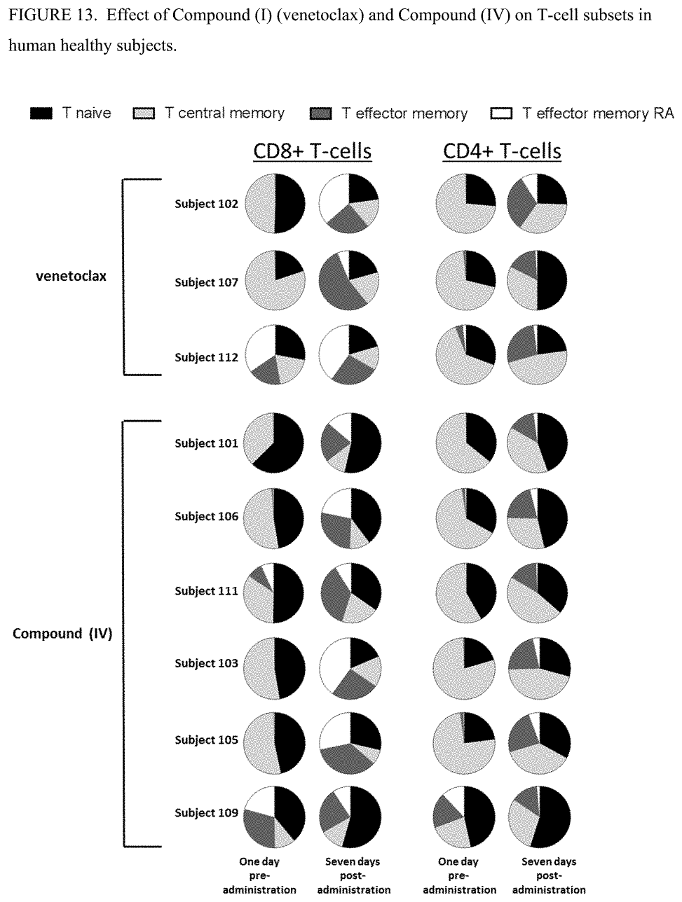

In a clinical study sponsored by AbbVie Inc. healthy volunteers were administered with one dose of 100 mg of Compound (I) (venetoclax) or a dose-equivalent of Compound (IV) in a solid formulation to deliver 100 mg of Compound (I). T-cell subsets in peripheral blood were measured one day pre- and seven days post-treatment by flow cytometry. In most of the tested subjects the fraction of CD4+ and CD8+ T effector memory and terminally differentiated effector memory cells increased, the proportion of CD8+ naïve T-cells has remained unchanged or decreased, and the proportion of CD4+ and CD8+ central memory T cells has decreased following treatment with either agent (Table 7 and FIG. 13). This data in human subjects underscore our findings in pre-clinical models that venetoclax mostly affects naïve T-cells, and leads to an increase of effector memory cells.

TABLE 7
Effect of Compound (I) (venetoclax) and Compound
(IV) on T-cell subsets in human healthy subjects

| | | CD8+ T-Cells | | | | CD4+ T-Cells | | | |
|---|---|---|---|---|---|---|---|---|---|
| | Subject # | TN | TCM | TEM | TEMRA | TN | TCM | TEM | TEMRA |
| Compound (I) | 102 | ↓ | ↓ | ↑ | ↑ | ≈ | ↓ | ↑ | ↑ |
| | 107 | ≈ | ↓ | ↑ | ↑ | ↑ | ↓ | ↑ | ≈ |
| | 112 | ≈ | ↓ | ↑ | ≈ | ↓ | ↓ | ↑ | ≈ |
| Compound (IV) | 101 | ↓ | ↓ | ↑ | ↑ | ↑ | ↓ | ↑ | ↑ |
| | 106 | ↓ | ↓ | ↑ | ↑ | ↑ | ↓ | ↑ | ↑ |
| | 111 | ↓ | ↓ | ↑ | ≈ | ↓ | ↓ | ↑ | ≈ |
| | 103 | ↓ | ↓ | ↑ | ↑ | ≈ | ↓ | ↑ | ↑ |
| | 105 | ↓ | ↓ | ↑ | ↑ | ≈ | ↓ | ↑ | ↑ |
| | 109 | ↑ | ≈ | ≈ | ↑ | ↑ | ↑ | ≈ | ↓ |

↑ increase
↓ decrease
≈ no change

The data presented here demonstrate that, not only does venetoclax not antagonize the immune-mediated anti-tumor activity of anti-PD-1 or anti-PD-L1 antibodies as originally believed, it enhances their efficacy in syngeneic/immunocompetent mouse models. Furthermore, as demonstrated by tumor re-challenge experiments, venetoclax treatment not only does not interfere with the establishment of anti-tumor memory, subjects responsive to venetoclax treatment develop anti-tumor memory. Data generated in multiple functional human T-cell activation assays (MLR and CMV recall) indicate that the beneficial effect of venetoclax stems from its ability to enhance activation of surviving T-cells. Moreover, the data showed unexpected anti-tumor activity against solid tumors. Taken as a whole, these unexpected findings indicate that venetoclax has previously unanticipated uses in the treatment of cancers, including solid tumors.

We claim:

1. A method for the treatment of a hematologic cancer in a subject who is in need thereof, comprising administering to the subject an effective amount of either an anti-PD-1 (programmed cell death protein-1) antibody or an anti-PD-L1 (programmed death ligand-1) antibody in combination with an effective amount of a selective BCL-2 inhibitor or a prodrug or pharmaceutically acceptable salt thereof.

2. The method of claim 1 for the treatment of a hematologic cancer in a subject who is in need thereof, comprising administering to the subject an effective amount of either an anti-PD-1 (programmed cell death protein-1) antibody or an anti-PD-L1 (programmed death ligand-1) antibody in combination with an effective amount of Compound (I), Compound (II), Compound (III) or Compound (IV) or a pharmaceutically acceptable salt thereof.

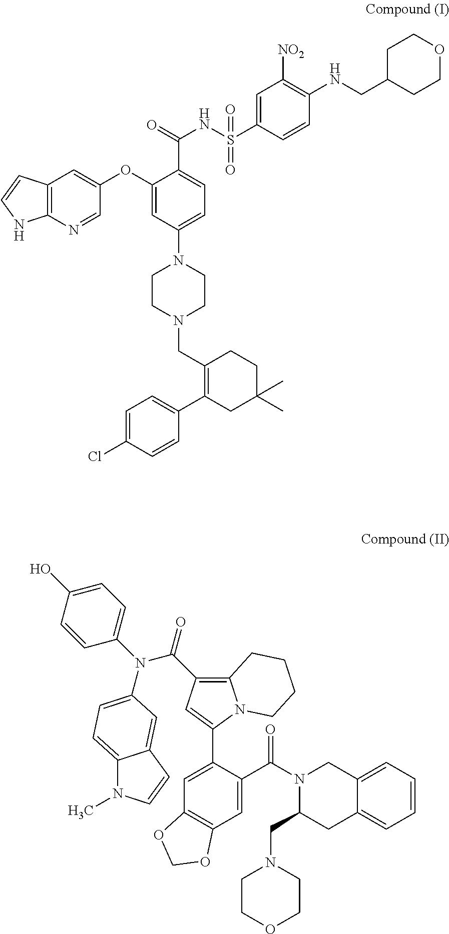 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1 ; Cn1ccc2cc(N(C(=O)c3cc(-c4cc5c(cc4C(=O)N4Cc6ccccc6C[C@H]4CN4CCOCC4)OCO5)n4c3CCCC4)c3ccc(O)cc3)ccc21]

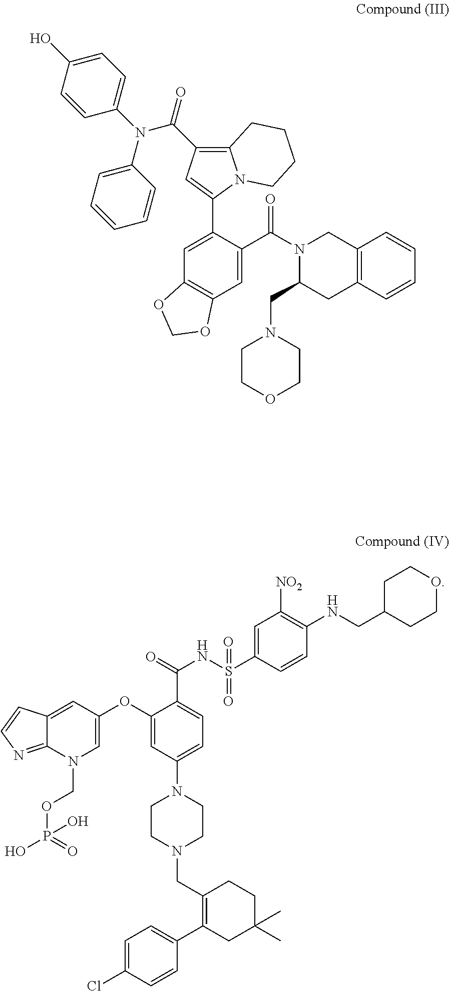 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1 ; CC1(C)CCC(CN2CCN(c3ccc(C(=O)NS(=O)(=O)c4ccc(NCC5CCOCC5)c([N+](=O)[O-])c4)c(Oc4cc5ccnc-5n(COP(=O)(O)O)c4)c3)CC2)=C(c2ccc(Cl)cc2)C1]

3. The method of claim 1 for the treatment of a hematologic cancer in a subject who is in need thereof, comprising administering to the subject an effective amount of either an anti-PD-1 (programmed cell death protein-1) antibody or an anti-PD-L1 (programmed death ligand-1) antibody in combination with an effective amount of Compound (I), wherein Compound (I) is venetoclax or a pharmaceutically acceptable salt thereof.

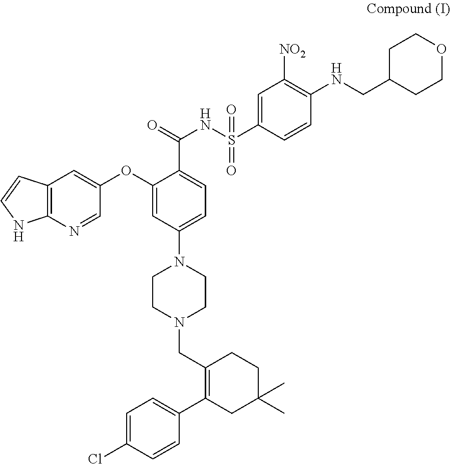 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

4. The method of claim 1 for the treatment of a hematologic cancer in a subject who is in need thereof, comprising administering to the subject an effective amount of an anti-PD-1 (programmed cell death protein-1) antibody in combination with an effective amount of Compound (I), wherein Compound (I) is venetoclax or a pharmaceutically acceptable salt thereof.

5. The method of claim 4, wherein the hematologic cancer is acute lymphoblastic leukemia (ALL), acute myeloid leukemia (AML), chronic myeloid leukemia (CML), chronic lymphocytic leukemia (CLL), small lymphocytic lymphoma (SLL), acute monocytic leukemia (AMoL), Hodgkin's lymphoma, non-Hodgkin lymphomas (NHL), multiple myeloma, or myelodysplastic syndrome (MDS).

6. The method of claim 4, wherein the anti-PD-1 antibody is pembrolizumab, nivolumab, cemiplimab, or ABBV-181.

7. The method of claim 4, wherein the anti-PD-1 antibody has a heavy chain sequence comprising SEQ ID NO: 7 and a light chain sequence comprising SEQ ID NO: 8.

8. The method of claim 4, wherein the anti-PD-1 antibody has a heavy chain sequence comprising SEQ ID NO: 9 and a light chain sequence comprising SEQ ID NO: 10.

9. The method of claim 4, wherein the anti-PD-1 antibody has a heavy chain sequence comprising SEQ ID NO: 1 and a light chain sequence comprising SEQ ID NO: 2.

10. The method of claim 4, wherein the anti-PD-1 antibody has a heavy chain sequence comprising SEQ ID NO: 54 and a light chain sequence comprising SEQ ID NO: 55.

11. The method of claim 1 for the treatment of a hematologic cancer in a subject who is in need thereof, comprising administering to the subject an effective amount of an anti-PD-L1 (programmed death ligand-1) antibody in combination with an effective amount of Compound (I), wherein Compound (I) is venetoclax or a pharmaceutically acceptable salt thereof.

12. The method of claim 11, wherein the hematologic cancer is acute lymphoblastic leukemia (ALL), acute myeloid leukemia (AML), chronic myeloid leukemia (CML), chronic lymphocytic leukemia (CLL), small lymphocytic lymphoma (SLL), acute monocytic leukemia (AMoL), Hodgkin's lymphoma, non-Hodgkin lymphomas (NHL), multiple myeloma, or myelodysplastic syndrome (MDS).

13. The method of claim 11, wherein the anti-PD-L1 antibody is atezolizumab, avelumab, or durvalumab.

14. The method of claim 11, wherein the anti-PD-L1 antibody has a heavy chain sequence comprising SEQ ID NO: 11 and a light chain sequence comprising SEQ ID NO: 12.

15. The method of claim 11, wherein the anti-PD-L1 antibody has a heavy chain sequence comprising SEQ ID NO: 13 and a light chain sequence comprising SEQ ID NO: 14.

16. The method of claim 11, wherein the anti-PD-L1 antibody has a heavy chain sequence comprising SEQ ID NO: 15 and a light chain sequence comprising SEQ ID NO: 16.

17. Methods for the treatment of solid tumor cancer in a subject who is in need thereof, comprising administering to the subject an effective amount of either an anti-PD-1 (programmed cell death protein-1) antibody or an anti-PD-L1 (programmed death ligand-1) antibody in combination with an effective amount of a selective BCL-2 inhibitor or pharmaceutically acceptable salt thereof.

18. The method of claim 17 for the treatment of a solid tumor cancer in a subject who is in need thereof, comprising administering to the subject an effective amount of either an anti-PD-1 (programmed cell death protein-1) antibody or an anti-PD-L1 (programmed death ligand-1) antibody in combination with an effective amount of an effective amount of Compound (I), Compound (II), Compound (III), or Compound (IV) or a pharmaceutically acceptable salt thereof.

19. The method of claim 17 for the treatment of a solid tumor cancer in a subject who is in need thereof, comprising administering to the subject an effective amount of either an anti-PD-1 (programmed cell death protein-1) antibody or an anti-PD-L1 (programmed death ligand-1) antibody in combination with an effective amount of Compound (I), wherein Compound (I) is venetoclax or a pharmaceutically acceptable salt thereof.

20. The method of claim 17 for the treatment of a solid tumor cancer in a subject who is in need thereof, comprising administering to the subject an effective amount of an anti-PD-1 (programmed cell death protein-1) antibody in combination with an effective amount of Compound (I), wherein Compound (I) is venetoclax or a pharmaceutically acceptable salt thereof.

21. The method of claim 20, wherein the solid tumor cancer is non-small cell lung cancer, gastric cancer, melanoma, microsatellite instability-high cancer, head and neck squamous cell cancer, metastatic cutaneous squamous cell carcinoma, locally advanced cutaneous squamous-cell carcinoma, urothelial bladder cancer, colorectal cancer, liver cancer, renal cell carcinoma, breast cancer, cervical cancer or Merkel cell carcinoma.

22. The method of claim 20, wherein the anti-PD-1 antibody is pembrolizumab, nivolumab, cemiplimab, or ABBV-181.

23. The method of claim 20, wherein the anti-PD-1 antibody has a heavy chain sequence comprising SEQ ID NO: 7 and a light chain sequence comprising SEQ ID NO: 8.

24. The method of claim 20, wherein the anti-PD-1 antibody has a heavy chain sequence comprising SEQ ID NO: 9 and a light chain sequence comprising SEQ ID NO: 10.

25. The method of claim 20, wherein the anti-PD-1 antibody has a heavy chain sequence comprising SEQ ID NO: 1 and a light chain sequence comprising SEQ ID NO: 2.

26. The method of claim 20, wherein the anti-PD-1 antibody has a heavy chain sequence comprising SEQ ID NO: 54 and a light chain sequence comprising SEQ ID NO: 55.

27. The method of claim 17 for the treatment of a solid tumor cancer in a subject who is in need thereof, comprising administering to the subject an effective amount of an anti-PD-L1 (programmed death ligand-1) antibody in combination with an effective amount of Compound (I), wherein Compound (I) is venetoclax or a pharmaceutically acceptable salt thereof.

28. The method of claim 27, wherein the solid tumor cancer is non-small cell lung cancer, gastric cancer, melanoma, microsatellite instability-high cancer, head and neck squamous cell cancer, metastatic cutaneous squamous cell carcinoma, locally advanced cutaneous squamous-cell carcinoma, urothelial bladder cancer, colorectal cancer, liver cancer, renal cell carcinoma, breast cancer, cervical cancer or Merkel cell carcinoma.

29. The method of claim 27, wherein the anti-PD-L1 antibody is atezolizumab, avelumab, or durvalumab.

30. The method of claim 27, wherein the anti-PD-L1 antibody has a heavy chain sequence comprising SEQ ID NO: 11 and a light chain sequence comprising SEQ ID NO: 12.

31. The method of claim 27, wherein the anti-PD-L1 antibody has a heavy chain sequence comprising SEQ ID NO: 13 and a light chain sequence comprising SEQ ID NO: 14.

32. The method of claim 27, wherein the anti-PD-L1 antibody has a heavy chain sequence comprising SEQ ID NO: 15 and a light chain sequence comprising SEQ ID NO: 16.
